(19) United States
(12) Patent Application Publication
(10) Pub. No.: US 2009/0208493 A1
(43) Pub. Date: Aug. 20, 2009

(54) Compounds and methods for the selective inhibition of ABCB1, ABCC1 and ABCG2 transporters and the treatment of cancers, especially drug resistant cancers and high throughput flow cytometry assay to detect selective inhibitors

(75) Inventors: Richard S. Larson, Albuquerque, NM (US); Larry A. Sklar, Albuquerque, NM (US); Bruce S. Edwards, Albuquerque, NM (US); Irena D. Ivnitski-Steele, Albuquerque, NM (US); Tudor I. Oprea, Albuquerque, NM (US); Debbie M. Lovato, Albuquerque, NM (US); Hadya M. Khawaja, Albuquerque, NM (US); Stuart S. Winter, Albuquerque, NM (US); Susan M. Young, Albuquerque, NM (US)

Correspondence Address: COLEMAN SUDOL SAPONE, P.C., 714 COLORADO AVENUE, BRIDGE PORT, CT, 06605-1601, US

(73) Assignee: STC. UNM, Albuquerque, NM (US)

(21) Appl. No.: 12/315,132

(22) Filed: Nov. 28, 2008

Related U.S. Application Data
(60) Provisional application No. 61/004,342, filed on Nov. 27, 2007.
(60) Provisional application No. 61/124,377, filed on Apr. 16, 2008.
(60) Provisional application No. 61/131,214, filed on Jun. 6, 2008.

(51) Int. Cl. A61K 38/13; A61K 33/24; A61K 39/395; A61K 31/57; G01N 33/50; A61P 35/00

(52) U.S. Cl. 424/133.1; 514/11; 424/649; 514/179; 435/40.51

(57) ABSTRACT

Compounds disclosed which inhibit ABCB1 transporter protein are useful for treating diseases in which ABCB1 transporter protein mediates the disease state, including numerous cancers, including hematopoietic cancers, including various leukemias, especially T-lineage acute lymphoblastic leukemia, as well as cancerous tumors, especially forms which exhibit multiple drug resistance. Pharmaceutical compositions which comprise an inhibitor of ABCB1 transporter protein and at least one additional anticancer agent, optionally in combination with a pharmaceutically acceptable carrier, additive or excipient are another aspect of the present invention. A flow cytometry based, high-throughput screening (HST) assay that quantifies ABCB1 efflux is also disclosed. Methods of identifying inhibitors of ABCB1, ABCG2 and ABCC1 transporter proteins are also disclosed.

### RELATED APPLICATIONS AND FEDERALLY SPONSORED RESEARCH

This application claims the benefit of priority of U.S. provisional application Ser. No. 61/004,342, filed Nov. 27, 2007, entitled “Compounds and Methods for the Inhibition of ABCB1 and the Treatment of Cancers”, U.S. provisional application Ser. No. 61/124,377, filed Apr. 16, 2008, entitled “High throughput flow cytometry assay to detect selective inhibitors of ABCB1, ABCC1 and ABCG2 transporters” and U.S. provisional application Ser. No. 61/131,214, filed Jun. 6, 2008, entitled “Novel ABCB1 inhibitors”, each of said applications being incorporated by reference in their entirety herein.

The present invention was made with government support under Grant No. R01 CA114589-01, awarded by the National Institutes of Health (NIH). Consequently, the Government has certain rights in this invention.

### FIELD OF THE INVENTION

The present invention relates to compounds which inhibit ABCB1 transporter protein. The compounds and methods are useful for treating diseases in which ABCB1 transporter protein mediates the disease state, and in particular, cancer, especially drug resistant (DR) and multiple drug resistant (MDR) cancer. The treatment of cancer, including the treating of various leukemias, especially T-lineage acute lymphoblastic leukemia, especially forms which are multiple drug resistant, are important features of the present invention. Pharmaceutical compositions which comprise an inhibitor of ABCB1 transporter protein and at least one additional anticancer agent, optionally in combination with a pharmaceutically acceptable carrier, additive or excipient are additional aspects of the present invention.

This invention also relates generally to high throughput flow cytometry assays for identification of molecular inhibitors of ATP binding cassette (ABC) transporters, more particularly ABCB1, ABCC1 and ABCG2 transporters.

### BACKGROUND OF THE INVENTION

Multiple Drug Resistance (MDR) is a process that occurs when cancer cells become resistant to an array of chemotherapeutic agents.¹ The phenomenon of MDR is largely mediated by a class of 170-kD proteins that have 12 transmembrane domains and 2 adenosine triphosphate (ATP) binding cassette (ABC) sites.² Although more than 48 members of the ABC transporter superfamily have been identified, relatively few, such as ABCB1, ABCC1 and ABCG2, appear to have the greatest potential clinical impact on therapy resistance.³ Of clinical importance, many intrinsically chemotherapy-resistant malignancies overexpress ABCB1 at the time of diagnosis.¹ Moreover, in patients with hematological malignancies, including T-lineage acute lymphoblastic leukemia (T-ALL), ABCB1 is often upregulated at the time of recurrence:^{4,5} The increased expression of ABCB1 in human cancer has important consequences for an individual's susceptibility to certain drug-induced side effects and treatment efficacy, and as a result, chemosensitizing agents have been sought to block the efflux potential of ABCB1.¹

The first. ABCB1 efflux-blocking agent discovered was verapamil, a drug that belongs to a family of calcium ion influx inhibitors. Following this discovery, numerous other agents were found to block the ABCB1 drug transporter, including cyclosporine A (CSA),^{6′} a cyophilin-binding immunosuppressive agent. In phase I/II clinical trials, the CSA-induced complications of vomiting and confusion were generally unmanageable. However, more serious complications of hypertension and renal insufficiency limited the use of this drug in cancer patients and called for the identification of ABCBI transport inhibitors having less toxicity.

High-throughput screening (HTS) allows the testing of numerous cellular targets against a wide variety of potentially valuable compounds. To address the need to identify target compounds that block ABCB1-mediated drug transport, we employed HyperCyt⁷ to screen a library of more than 880 off-patent drugs for potential use as chemosensitizing agents. The inventors developed a chemoresistant T-ALL cell line that could be tested for ABCB I-reversal agents using J-aggregate-forming lipophilic cation 5,5′,6,6′-tetrachloro-1,1,′3,3′-tetraethylbenzimidazolcarbocyanine iodide (JC-1),^{8,9} a cationic dye that exhibits differential fluorescence based on its intracellular concentrations. The initial screen identified 19 target compounds, and on the basis of a published record of safe use in humans, the inventors investigated 11 compounds for further analysis. On the basis of an unfavorable in vitro toxicity profile, we excluded 4 drugs, leaving 7 compounds for further analysis. These 7 drugs have diverse structural and functional classifications and include cation channel-blocking agents, an angiotensin-converting enzyme (ACE) inhibitor, an imidazole, and an immunosuppressant. Through HTS for ABCB I-reversal agents, we also identified mometasone furoate as a drug that merits further exploration.

### SUMMARY OF THE INVENTION

The present invention relates to the use of ABCB1 inhibitors for the treatment of cancer, in particular, drug resistant (DR) cancer, and more particularly, to multiple drug resistant (MDR) cancer. The present invention also relates to pharmaceutical compositions comprising an ABCB1 inhibitor according to the present invention in combination with an anticancer agent, optionally in combination with a pharmaceutically acceptable carrier, additive and/or excipient and the use of these compositions in the treatment of cancer, especially including drug resistant and multiple drug resistant forms of cancer. The present invention also relates to methods for reducing the likelihood of metastasis of cancer, the recurrence of cancer after a patient has been in remission and the likelihood that a cancer will develop drug resistance, including multiple drug resistance during therapy of a patient. One or more of the ABCB1 inhibitors which are preferably used in the present invention are selected from the group consisting of bepridil, rescinnamine, nicardipine, propafenone, ketoconazole, cyclosporine A, loxapine, pimozide, acacetin, mometasone furoate or its active 6-β-hydroxy metabolite, or a pharmaceutically acceptable salt thereof. Preferred anticancer agents which are used in the present invention are those in which ABCB1 transporter are implicated in drug resistance and include the anthracyclines (daunorubin, doxorubicin, epirubicin, idarubicin, and valrubicin), the vinca alkaloids (vincristine, vinblastine, vindesine and vinorelbine), the taxanes (paclitaxel or taxol, and docetoxel or taxotere), epidopodophyllotoxins (etoposide or VP-16 and tenoposide), nelarabine and imatinib, among others.

As discussed above, the dose-limiting toxicities of many agents which may be used to treat multiple drug resistant (MDR) cancers have restricted their use in clinical trials, and consequently, more effective and clinically applicable agents remain to be identified. The inventors have developed a T-ALL cell line that overexpresses ABCB1 and exhibits multiple drug resistance (MDR) to daunorubicin, prednisolone, and vincristine, but not L-asparaginase. The MDR of the cells can be reversed by suppression of ABCB1 expression with siRNA or 5 μM cyclosporine (CSA). Using this cell line, a flow cytometry based, high-throughput screening assay was developed that quantifies ABCB1 efflux using the fluorescent probe JC-1.⁹ A library of drugs was screened for their ability to inhibit ABCB1 efflux at concentrations of 4 μM, and 19 compounds were identified, including CSA. Based on a published record of safe internal use in humans, 11 compounds (which appear in Table 1, below) were retained for further analysis. 3 of these compounds were originally described and used as calcium channel blockers, 1 was described and used as a sodium channel blocker, 1 as an ACE inhibitor, 1 as a dopamine uptake inhibitor, 1 as a Topoisomerase II inhibitor, 2 as steroidal agents, 1 as an antifungal and 1 as an immunosuppressant. The inventors determined the 50% inhibitory concentration (IC₅₀) of ABCB 1 efflux, the efflux reversal concentration of drug that rescued DNR-induced T-ALL cell death (ECᵢev₅₀), and the corresponding in vitro toxic dose in 50% of treated T-ALL cells (TD54) (See Table 1). With the exception of CSA, none of these 10 compounds have been previously described as ABCB1 inhibitors. These compounds are useful for the treatment of disease states and/or conditions which are modulated through ABCB1 transporter protein including for example, cancer, including MDR cancer. Of the 11 identified, 7 which are described hereinbelow are preferred compounds for use in the treatment of cancers, especially drug resistant cancers as otherwise described herein.

The eleven compounds, which are selected from the group consisting of bepridil, lidoflazine, nicardipine, propafenone, rescinnamine, GBR 12909, ellipticine, hexestrol, mometasone furoate or its active 6β-hydroxy metabolite, ketoconazole and cyclosporin A (preferably, the less toxic compounds within this group namely, bepridil, nicardipine, propafenone, rescinnamine, ketoconazole, cyclosporine A, mometasone furoate, its active 6β-hydroxy metabolite, as well as the separately discovered loxapine, pimozide or acacetin and mixtures thereof), or their pharmaceutically acceptable salts and mixtures thereof, are useful as chemosensitizers, as compounds which function as inhibitors of ABCB1 transport protein, and/or as lead compounds for development of improved ABCB1 inhibitors in numerous cancers, as described in greater detail herein. Thus, the present invention is directed to methods of inhibiting (directly or indirectly) ABCB1 transport protein in a patient, to treating cancers, especially including drug resistant (DR) and multiple drug resistant (MDR) cancers in a patient, to reducing the likelihood of metastasis of cancers, to reducing the likelihood that a cancer will become drug resistant and/or multiple drug resistant during the course of therapy for that cancer and to reducing the likelihood of a recurrence of cancer after remission. Separately, the present invention is directed to methods for identifying other direct and indirect inhibitors of ABCB1, ABCC1 and ABCG2 transporter proteins. In addition to the treatment or reducing the likelihood of cancer the compounds which have been identified pursuant to the present invention may be used as lead compounds for providing novel compounds which evidence greater activity, including in treating cancer, especially drug resistant and multiple drug resistant (MDR) forms of cancer.

TABLE 1
Drugs that inhibit ABCB1

| | | Chemoᵣₑᵥ₅₀ | |
|---|---|---|---|
| Drug | IC_{5O} (SD) | (SD) | TD₅₀ (SD) |
| Bepridil HCL | 7.4 μM (0.95) | 3.3 μM (2.3) | 24.54 μM (27.2) |
| Lidoflazine | 5.4 μM (0.55) | 3.1 μM (1.7) | 15.1 μM |
| Nicardipine HCL | 0.76 μM (018) | 2.6 μM (1.1) | 19.4 μM (5.0) |
| Propafenone | 19.3 μM (2.1) | 4.9 μM (1.3) | 27.5 μM (5.9) |
| HCL | | | |
| Rescinnamine | 1.53 μM (0.5) | 1.0 μM (0.5) | 10.0 μM (6.4) |
| GBR 12909 HCL | 4.6 μM (0.28) | 2.1 μM (0) | 5.3 μM |
| Ellipticine | 0.99 μM (0.014) | 1.8 μM (0.92) | 3.8 μM |
| Hexestrol | 8.3 μM (3.2) | 12.8 μM (3.3) | 151 μM |
| Mometasone | 0.74 μM (0.13) | 1.4 μM (0.28) | 30.4 μM |
| Furoate | | | |
| Ketoconazole | 2.18 μM (0.45) | 3.7 μM (0.99) | 37.9 μM (0.21) |
| Cyclosporin A | 0.31 (0.087) | 0.63 μM (0.01) | 3.4 μM (2.34) |

IC₅₀ 50% - inhibition of drug efflux determined in high-throughput screen;
Chemoᵣₑᵥ₅₀ - 50% of reversal of chemotherapeutic effect determined in in vitro screen;
TD₅₀ - 50% toxic dose determined in in vitro screen.

Note that lidoflazine, GBR 12909, ellipticine and hexestrol, because of less than desirable in vitro therapeutic indices (a function of activity, toxicity or a combination of both), are less preferred for use in the present invention for the treatment of cancer, but represent viable lead compounds from which combinatorial approaches to increasing anticancer activity and reducing toxicity may be provided.

The present invention may be used to treat any disease in which ABCB1 transporter protein is overexpessed, including cancer, especially drug resistant cancers which overexpress ABCB1. While numerous cancers may be treated using the compositions and methods according to the present invention, cancers which are shown to be particularly responsive to therapeutic methods according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML, among others. The method shows particular effect in treating virtually any cancer in which drug resistance and/or multiple drug resistance is a potential factor by virtue of an overexpression of ABCB1 transporter protein. A large number of cancers may be treated using the present invention, including various leukemias and solid tumors, among others, as otherwise described herein.
In the present invention, the present compounds and compositions may be used to treat diseases which are mediated through overexpression of ABCB1 transporter protein. The methods of the invention are particularly suited to the treatment of cancers, including drug resistant and multiple drug resistant cancers, especially including cancers which overexpress ABCB1 transporter protein. Numerous cancers may be treated using the present invention including hematopoietic neoplasms, including Hodgkin's disease, non-Hodgkin's lymphoma, leukemias, including non-acute and acute leukemias, such as acute myelogenous leukemia, acute lymphocytic leukemia, acute promyelocytic leukemia (APL), acute T-cell lymphoblastic leukemia, R-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia, stem cell leukemia and metastasis of these diseases. Other cancers which may be treated according to the present invention include, for example, stomach (especially including gastric stromal cells), colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, skin cancer, including melanoma and non-melanoma, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among numerous others. As indicated above, particularly responsive cancers to the methods according to the present invention include T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML, breast cancer, Ewing's sarcoma, osteosarcoma and undifferentiated high-grade sarcomas, among others.

Turning to an identification method aspect of the present invention, the present invention also is directed to methods of identifying inhibitors of ABCB1, ABCC1 and ABCG2 transporter proteins. Identification of new transporter-specific inhibitors may assist in better understanding transporters function and to develop effective clinically relevant compounds aimed at overcoming multiple drug resistance in cancers. The present invention therefore also relates to methods of identifying inhibitors of ABCB1, ABCC1 and ABCG2 transporter proteins and the use of the inhibitors identified in the treatment of conditions or disease states in which multidrug resistance is implicated, especially including cancer. Also described is a combination assay which combines a ABCG2 assay with an ABCB1 assay to allow immediate evaluation of transporter specificity of any inhibitors identified.

In this assay method described herein, high throughput screening (HTS) allows the testing of numerous cellular targets against a wide variety of potentially valuable compounds. To address the need to identify target compounds that block ABCB1-mediated drug transport, the inventors employ HyperCyt⁷ to screen a large library of compounds for potential use as chemo-sensitizing agents.

### BRIEF DESCRIPTION OF THE FIGURES

FIGS. 1A-1D are graphs showing that fluorescent substrates allow measurement of transporter activity. FIG. 1A shows fluorescence intensity of a JC1 substrate in parental Igrov 1 cells (dotted line), and in IgMXP3 cells in the absence (bold line) and presence (grey fill) of the ABCG2 transporter inhibitor Novobiocin. FIG. 1B shows fluorescence intensity of CaAM substrate in parental SupT1 cells (dotted line), and in SupT1-Vin150 cells in the absence (bold line) and presence (grey fill) of the ABCC1 transporter inhibitor MK571. FIG. 1C shows fluorescence intensity of CaAM substrate in parental Jurkat cells (dotted line), and in Jurkat-DNR cells in the absence (bold line) or presence (grey fill) of the ABCB1 pump inhibitor Cyclosporin A. FIG. 1D shows fluorescence intensity of CaAM substrate in parental CCRF-CEM cells (dotted line), and in CCRF-ADR cells in the absence (bold-line) or presence (grey fill) of Cyclosporin A.

FIGS. 2A-2F are graphs demonstrating a high-throughput-flow cytometric analysis of ABC transporter activity. FIGS. 2A-2C show results of a JC1 duplex assay in which IgMXP3 cells (ABCG2, dim red/FL8 fluorescent) and Jurkat DNR cells (ABCB1, bright red/FL8 fluorescent) are enclosed in separate electronic gates (FIG. 2A, circles) to allow separate analysis of the green JC1 fluorescence intensity response (FL1) of cells sampled from a 384 well plate (FIGS. 2B and 2C, respectively). In the plots of Time vs. FL1 in FIGS. 2B and 2C, each discrete cluster of dots represents cells sampled from a single well. Arrows indicate cells from negative vehicle control wells (N), wells containing the positive control compound Nicardipine (P), and the well containing Lasalocid (Las), a compound that resulted in a significant assay response for both transporters. FIGS. 2D-2F show results of a CaAM duplex assay. SupT1 Vin cells (ABCC1, dim red/FL8 fluorescent) and CCRF-ADR cells (ABCB1, bright red/FL8 fluorescent) are enclosed in gates (FIG. 2D) to allow separate analysis of the green CaAM fluorescence intensity response (FL1) of cells sampled from a 384 well plate (FIGS. 2E and 2F, respectively). Arrows indicate cells from negative and positive control wells as above, and cells treated with Loxapine (Lox), a compound that selectively produced a significant assay response in ABCC1-expressing cells. In all the time resolved dot plots (FIGS. 2B, 2C, 2E, 2F) only a 30 to 40 s segment of the full 384 well data acquisition sequence (˜11 min total) are illustrated.

FIGS. 3A-3F are graphs depicting a dose response analysis of pump inhibition and chemosensitivity profile changes promoted by representative compounds. Pimozide (FIG. 3A), metergoline (FIGS. 3B) and (3C) nicardipine were tested for the ability to inhibit activity of ABCB1 (squares), ABCG2 (triangles) and ABCC1 (diamonds) transporters. Each compound was tested over a concentration range of 4 nM to 25 μM. Illustrated ABCB1 results were from analysis of CCRF-Adr cells using CaAM substrate. FIG. 3D shows cell viability of ABBC1-expressing SupT1-Vin cells over a range of vincristin (VIN) concentration in the presence (triangles) and absence (filled circles) of 1.6 μM pimozide. FIG. 3E depicts cell viability of ABCB1-expressing Jurkat DNR cells over a range of daunorubicin (DNR) concentration in the presence (triangles) and absence (filled circles) of 3.1 μM ivermectin. FIG. 3F depicts cell viability of ABCG2-expressing IgMXP3 cells over a range of mitoxantrone (MTX) concentration in the presence (triangles) and absence (filled circles) of 3.1 μM niclosamide. In each assay the chemosensitivity profile of appropriate parental cells that did not express transporters was also determined (open circles). The concentration of each cytotoxic drug ranged from 70 nM to 50 μM.

FIGS. 4, 5A, 5B, and 6A-6D are graphs pertinent to how Jurkat-cells with multi-drug resistance can be used to screen off-patent drugs. FIG. 4 is a bar graph of RMA-normalized gene profiling in wild-type and DNR-resistant Jurkat cells, showing that ABCB1 was 591-fold (SD±274) up-regulated in the drug-resistant cell line. ABBC1 and ABCG2 were upregulated less than 1.5 fold in response to DNR. FIG. 5A is a graph showing that parental (therapy sensitive) Jurkat cells did not express ABCB1 (solid peak: murine IgG₂ₐ PE isotype mAbs; dotted solid peak: PE-conjugated ABCB1 mAb). FIG. 5B is a graph showing that therapy-resistant Jurkat cells express increased levels of ABCB1, as demonstrated by a 2 log(₁₀) shift in peak fluorescence intensity (solid peak[left]: murine Ig/G₂ₐ PE isotype mAbs; bold solid peak [right], PE-conjugated ABCB1 mAb). FIGS. 6A-6D are graphs showing that parental (therapy sensitive) Jurkat cells exhibit decreased IC₅₀ levels in comparison to cells that are therapy-resistant to DNR (FIG. 6A), VCR (FIG. 6B), and to a lesser degree, to PRED (FIG. 6C); but not to L-ASP (FIG. 6D.

FIGS. 7A-7C are graphs showing that JC-1 is effluxed from DNR-resistant Jurkat cells, but not in the presence of ABCB1-reversal agents. FIG. 7A is a graph showing that parental Jurkat cells (vertical lines), do not efflux the JC-1 probe either with Cyclosporin (right up-slanted) or without Cyclosporin (left down-slanted). FIG. 7B shows therapy-resistant Jurkat (vertical lines) can exclude JC-1 (left down-slanted), but not in the presence of Cyclosporin (right up-slanted), an inhibitor of ABCB1-mediated efflux. FIG. 7C is a test result graph showing that a JC-1 carbocyanine liquid-forming probe displays either red (597 nm) or green (537 nm) fluorescence that is dependent on high or low intracellular concentrations, respectively. Fluorescence intensity is measured along y-axis and sample number along the x-axis. Samples with higher intracellular JC-1 concentrations, and therefore higher fluorescence intensity (block arrows), are considered “hits”.

FIGS. 8A-8C are graphs showing the determination of JC-1 IC₅₀ curves for ABCB1 efflux inhibitors. FIG. 8A relates to identified cation channel blockers including nicardipine (first curve from left at 50% inhibition level), lidoflazine (second curve from left at 50% inhibition level), bepridil (third curve from left at 50% inhibition level) and propafenone (fourth curve from left at 50% inhibition level). FIG. 8B relates to intra-nuclear receptors/ligands including mometasone (first curve from left), ellipticine (second curve from left), and hexestrol (third curve from left). FIG. 8C relates to other classes identified including an immunosuppressive agent (cyclosporine; first curve from left at 50% inhibition level), an anti-fungal agent (ketoconazole; second curve from left at 50% inhibition level), a neuro-transmitter blocker (GBR12909; fourth curve from left at 50% inhibition level) and an inhibitor of angiotensin converting enzyme (rescinaminne; third curve from left at 50% inhibition level).

FIGS. 9A-9K are graphs of DNR reversal ranges for ABCB1-inhibitors maintained in culture for 7 days. For each reversal agent, cell survival is shown in the presence (-) or absence ( . . . ) of 100 nM DNR. A range in vitro therapeutic indices is observed for each ABCB1-reversal agent. Narrow ranges would indicate a high likelihood of toxicity at effective serum concentrations.

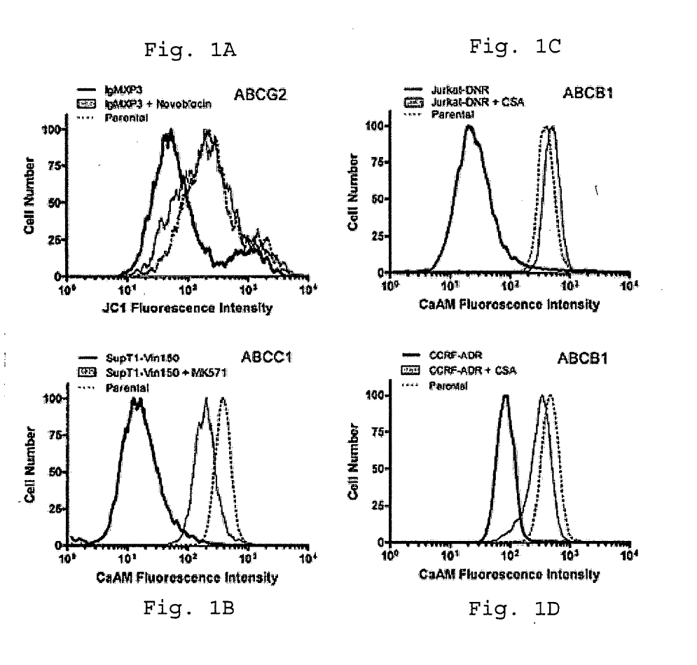

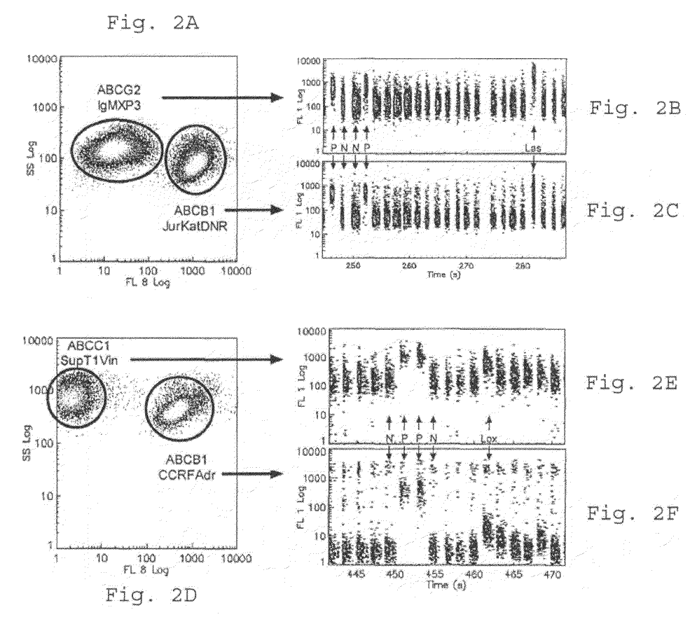

### DEFINITIONS

The following terms are used throughout the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

The term “patient” or “subject” is used throughout the specification to describe an animal, preferably a human, to whom treatment, including prophylactic treatment, with the compositions according to the present invention is provided. For treatment of those infections, conditions or disease states which are specific for a specific animal such as a human patient, the term patient refers to that specific animal.

The term “compound” is used herein to refer to any specific chemical compound disclosed herein. Within its use in context, the term generally refers to a single small molecule as disclosed herein, but in certain instances may also refer to stereoisomers and/or optical isomers (including racemic mixtures) of disclosed compounds. The term compound includes active metabolites of compounds and/or pharmaceutically active salts thereof.

The term “inhibitor” is used herein to refer to any compound which produces an inhibition of ABCB1 transporter protein by any mechanism, direct or indirect, whether it be by inhibition of the interaction of ABCB1 transporter protein with its intended receptor or other target or whether it be by inhibition of the expression of ABCB1 transporter protein.

The term “effective amount” is used throughout the specification to describe concentrations or amounts of compounds or other components which are used in amounts, within the context of their use, to produce an intended effect according to the present invention. The compound or component may be used to produce a favorable change in a disease or condition treated, whether that change is a remission, a favorable physiological result, a reversal or attenuation of a disease state or condition treated, the prevention or the reduction in the likelihood of a condition or disease-state occurring, depending upon the disease or condition treated. Where compounds are used in combination, each of the compounds is used in an effective amount, wherein an effective amount may include a synergistic amount. In many instances, the term effective amount refers to that amount which inhibits expression of ABCB1 and consequently, results in a dimunition of resistance to a therapeutic approach, to symptoms or results in an actual cure of a disease state such as cancer, which cancer may include drug resistant cancer, especially a multiple drug resistant (MDR) cancer, a cancer such as a leukemia or a cancerous tumor, especially including T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML, among others.

In the present invention all compounds are used in effective amounts to provide activity relevant to the use of the compound. In combination therapy, the ABCB1 inhibitor and the anticancer agent are both used in effective amounts. The amount of compound used in the present invention may vary according to the nature of the compound, the age and weight of the patient and numerous other factors which may influence the bioavailability and pharmacokinetics of the compound, the amount of compound which is administered to a patient generally ranges from about 0.001 mg/kg to about 50 mg/kg or more, about 0.5 mg/kg to about 25 mg/kg, about 0.1 to about 15 mg/kg, about 1 mg to about 10 mg/kg per day and otherwise described herein. The person of ordinary skill may easily recognize variations in dosage schedules or amounts to be made during the course of therapy.

The term “ABCB1 mediated disease” is used throughout the specification to describe a disease which is mediated through the action or overexpression of ABCB1 transporter protein or where the overexpression of ABCB1 transporter protein occurs in conjunction with the disease state. Diseases which may be treated according to the present invention include a cancerous disease state, in particular, a drug resistant cancer, a multiple drug resistant cancer, a leukemia or related hematopoietic cancer, including T-ALL and related leukemias, especially drug resistant (multiple) leukemias, such as T-ALL, and numerous cancerous tumors as otherwise described herein. These diseases may include any one or more of hematopoietic neoplasms and metastasis of such neoplasms, including Hodgkin's disease, non-Hodgkin's lymphoma, leukemias, including non-acute and acute leukemias, such as acute myelogenous leukemia, acute lymphocytic leukemia, acute promyelocytic leukemia (APL), acute T-cell lymphoblastic leukemia, T-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia. Other cancers, including cancerous tumors, which may be treated using the present invention include for example, stomach (especially including gastric stromal cells), colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, skin cancer, including melanoma and non-melanoma, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others. Additional cancers which may be particularly responsive to therapeutic methods according to the present invention include for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML, breast cancer, Ewing's sarcoma, osteosarcoma and undifferentiated high-grade sarcomas, among others.

The term “neoplasia” or “neoplasm” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and may invade surrounding tissues. As used herein, the term neoplasia/neoplasm is used to describe all cancerous disease states and embraces or encompasses the pathological process associated with cancer, including hematopoietic cancers, numerous cancerous tumors and their metastasis.

A “hematopoietic neoplasm” or “hematopoietic cancer” is a neoplasm or cancer of hematopoeitic cells of the blood or lymph system and includes disease states such as Hodgkin's disease, non-Hodgkin's lymphoma, leukemias, including non-acute and acute leukemias, such as acute myelogenous leukemia, acute lymphocytic leukemia, acute promyelocytic leukemia (APL), adult T-cell leukemia, T-lineage acute lymphoblastic leukemia (T-ALL), basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia, among others.

The present method may be used to treat all cancers, especially the above hematopoetic and tumorigenic cancers which exhibit an overexpression of ABCB1 transporter protein. While T-ALL and especially multiple drug resistant T-ALL are particularly relevant disease targets for the methods of the present invention, virtually any cancer which overexpresses ABCB1 transporter protein or where ABCB1 transporter protein is implicated in instilling drug resistance or multiple drug resistance to the cancer and/or tumor is an appropriate target of the present therapeutic methods and compositions according to the present invention. Cancers which are particularly response to the present invention include T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML, breast cancer, Ewing's sarcoma, osteosarcoma and undifferentiated high-grade sarcomas, among others. Other cancers which may be treated according to the present invention include for example, stomach (especially including gastric stromal cells), colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, skin cancer, including melanoma and non-melanoma, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, including drug resistant (DR) and multiple drug resistant (MDR) forms of each of these cancers.

The term “prophylactic” is used to describe the use of a compound described herein which reduces the likelihood of an occurrence of a condition or disease state in a patient or subject. The term “reducing the likelihood” refers to the fact that in a given population of patients, the present invention may be used to reduce the likelihood of an occurrence, recurrence or metastasis of disease in one or more patients within that population of all patients, rather than prevent, in all patients, the occurrence, recurrence or metastasis of a disease state.

The term “pharmaceutically acceptable” refers to a salt form or other derivative (such as an active metabolite or prodrug form) of the present compounds or a carrier, additive or excipient which is not unacceptably toxic to the subject to which it is administered.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascetic and solid tumors.

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “flow cytometry” is used herein to identify a well-known process for counting, examining, and sorting microscopic particles suspended in a stream of fluid. The process utilizes an optical and/or electronic apparatus, called a “flow cytometer,” to conduct simultaneous multiparametric analysis of the physical and/or chemical characteristics of single cells flowing through the apparatus. A flow cytometer has 5 main components, (1) tubing and flow control to carry and align the cells so that they pass single file through a light beam for sensing, (2) an optical system including a radiation source such as a mercury or xenon lamp or a laser, (3) a detector and analog-to-digital converter, (4) an amplification system, and (5) a computer for analysis of the signals. The present application identifies several flow cytometers by maker. The present invention may include the use of duplex high-throughput flow cytometry. Methods according to the present invention which are used to identify inhibitors of ABC transporter proteins (e.g., ABCB1, ABCC1, ABCG2) adapt flow cytometry using readily available teachings to provide high throughput analysis of compounds and their effects on the ABC transporter proteins.

### DETAILED DESCRIPTION OF THE INVENTION

The present disclosure provides novel small molecules that inhibit ABCB1 transport proteins in cancer disease states, especially hematopoietic cancers and cancerous tumors as otherwise described herewith especially including those which are drug resistant, especially those disease states which are drug resistant as a consequence of overexpression of ABCB1 transporter protein. Various cancers as otherwise described herein may be treated using the methods of the present invention, especially including cancers exhibiting multiple drug resistance. Particularly responsive cancers to the present methods include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML, breast cancer, Ewing's sarcoma, osteosarcoma and undifferentiated high-grade sarcomas, among others.

The present invention relates unexpected activity of compounds which are well known in the art, but have heretofore not been known to be inhibitors of ABCB1. Methods of making these compounds and incorporating these compounds into pharmaceutical compositions are well known in the art. Pharmaceutically acceptable salts prepared from the active compounds are readily prepared. The present invention is not limited in any way by the method of synthesis of compounds, but encompasses all small molecules otherwise identified that may be produced by any suitable method of synthesis. Compounds may be synthesized step-wise by first synthesizing various synthons and then condensing the synthons together to produce compounds according to the invention. The synthesis of compounds according to the present invention is well within the routine skill of the person of ordinary skill. If desired, intermediates and products may be purified by chromatography and/or recrystallization. Starting materials, intermediates and reagents are either commercially available or may be prepared by one skilled in the art using methods described in the relevant chemical literature. Most of the compounds which are used therapeutically in the present invention are known in the art, as are the methods of their synthesis.

The present invention includes, where applicable, the compositions comprising the pharmaceutically acceptable salts, in particular, acid or base addition salts of compounds of the present invention. The acids which are used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3naphthoate)]salts, among numerous others.

Pharmaceutically acceptable base addition salts may also be used to produce pharmaceutically acceptable salt forms of the compounds or derivatives according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium, zinc and magnesium), ammonium or water-soluble amine addition salts such as N-methylglucamine-(meglumine), and the lower alkanolammonium and other base salts of pharmaceutically acceptable organic amines, among others.

The compounds of the present invention may, in accordance with the invention, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present invention as sprays, mists, or aerosols for intra-nasal, intra-tracheal or pulmonary administration may also be used. The present invention therefore also is directed to pharmaceutical compositions comprising an effective amount of compound according to the present invention, optionally in combination with a pharmaceutically acceptable carrier, additive or excipient. Compounds according to the present invention may be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

The amount compound which is used in the present invention, whether that compound is an ABCB1 inhibitor or an anticancer compound is that amount effective within the context of the administration of the compound(s). A suitable oral dosage for a compound of the present invention would be in the range of about 0.01 mg to 10 g or more per day, preferably about 0.1 mg to about 1 g per day. In parenteral formulations, a suitable dosage unit may contain from about 0.1 to about 250-500 mg of said compounds, which may be administered continuously or from one to four times per day, whereas for topical administration, formulations containing 0.01 to 1% or more by weight active ingredient are preferred. It should be understood, however, that the dosage administered from patient to patient will vary and the dosage for any particular patient will depend upon the clinician's judgment, who will use as criteria for fixing a proper dosage the size and condition of the patient as well as the patient's response to the drug.

When the compounds of the present invention are to be administered by oral route, they may be administered as medicaments in the form of pharmaceutical preparations which contain them in association with a compatible pharmaceutical carrier material. Such carrier material can be an inert organic or inorganic carrier material suitable for oral administration. Examples of such carrier materials are water, gelatin, talc, starch, magnesium stearate, gum arabic, vegetable oils, polyalkylene-glycols, petroleum jelly and the like. Immediate release, intermediate release and sustained and/or controlled release formulations are contemplated by the present invention.

The pharmaceutical formulations/preparations according to the present invention can be prepared in a conventional manner and finished dosage forms can be solid dosage forms, for example, tablets, dragees, capsules, and other like oral dosage forms, or liquid dosage forms, for example solutions, suspensions, emulsions and the like.

The pharmaceutical formulations/preparations may be subjected to conventional pharmaceutical operations such as sterilization. Further, the pharmaceutical preparations may contain conventional additives and excipients such as preservatives, stabilizers, emulsifiers, flavor-improvers, wetting agents, buffers, salts for varying the osmotic pressure and the like. Solid carrier material which can be used include, for example, starch, lactose, mannitol, methyl cellulose, microcrystalline cellulose, talc, silica, dibasic calcium phosphate, and high molecular weight polymers (such as polyethylene glycol).

For parenteral use, a compound according to the present invention can be administered in an aqueous (saline) or non-aqueous solution, suspension or emulsion in a pharmaceutically acceptable oil or a mixture of liquids, which may contain bacteriostatic agents, antioxidants, preservatives, buffers or other solutes to render the solution isotonic with the blood, thickening agents, suspending agents or other pharmaceutically acceptable additives. Additives of this type include, for example, tartrate, citrate and acetate buffers, ethanol, propylene glycol, polyethylene glycol, complex formers (such as EDTA), antioxidants (such as sodium bisulfite, sodium metabisulfite, and ascorbic acid), high molecular weight polymers (such as liquid polyethylene oxides) for viscosity regulation and polyethylene derivatives of sorbitol anhydrides. Preservatives may also be added if necessary, such as benzoic acid, methyl or propyl paraben, benzalkonium chloride and other quaternary ammonium compounds.

The compounds of this invention may also be administered as solutions for nasal application and may contain in addition to the compounds of this invention suitable buffers, tonicity adjusters, microbial preservatives, antioxidants and viscosity-increasing agents in an aqueous vehicle. Examples of agents used to increase viscosity are polyvinyl alcohol, cellulose derivatives, polyvinylpyrrolidone, polysorbates or glycerin. Preservatives added may include benzalkonium chloride, chlorobutanol or phenylethyl alcohol, among numerous others.

In certain aspects according to the present invention, where various cancers are to be treated, the compounds may be co-administered with at least one other anti-cancer agent such as antimetabolites, Ara C, etoposide, doxorubicin, taxol, hydroxyurea, vincristine, cytoxan (cyclophosphamide) or mitomycin C, among numerous others, including topoisomerase I and topoisomerase II inhibitors, such as adriamycin, topotecan, campothecin and irinotecan, other agent such as gemcitabine and agents based upon campothecin and cisplatin. By “co-administer” it is meant that the present compounds are administered to a patient such that the present compounds as well as the co-administered compound may be found in the patient's bloodstream at the same time, regardless when the compounds are actually administered, including simultaneously. In many instances, the co-administration of the present compounds with traditional anticancer agents produces a synergistic (i.e., more than additive) result which is unexpected.

Additional compounds which may be used in combination with the compounds uncovered in the present invention include for example: adriamycin, anastrozole, arsenic trioxide, asparaginase, azacytidine, BCG Live, bevacizumab, bexarotene capsules, bexarotene gel, bleomycin, bortezombi, busulfan intravenous, busulfan oral, calusterone, campothecin, capecitabine, carboplatin, carmustine, carmustine with polifeprosan 20 implant, celecoxib, cetuximab, chlorambucil, cisplatin, cladribine, clofarabine, cyclophosphamide, cytarabine, cytoxan, cytarabine liposomal, dacarbazine, dactinomycin, actinomycin D, dalteparin sodium, darbepoetin alfa, dasatinib, daunorubicin liposomal, daunorubicin, daunomycin, decitabine, denileukin, denileukin diftitox, dexrazoxane, dexrazoxane, docetaxel, doxorubicin, doxorubicin liposomal, dromostanolone propionate, eculizumab, Elliott's B Solution, epirubicin, epirubicin hcl, epoetin alfa, erlotinib, estramustine, etoposide phosphate, etoposide VP-16, exemestane, fentanyl citrate, filgrastim, floxuridine (intraarterial), fludarabine, fluorouracil 5-FU, fulvestrant, gefitinib, gemcitabine, gemcitabine hcl, gemicitabine, gemtuzumab ozogamicin, goserelin acetate, goserelin acetate, histrelin acetate, hydroxyurea, ibritumomab tiuxetan, idarubicin, ifosfamide, imatinib mesylate, interferon alfa 2a, interferon alfa-2b, irinotecan, lapatinib ditosylate, lenalidomide, letrozole, leucovorin, leuprolide acetate, levamisole, lomustine CCNU, meclorethamine, nitrogen mustard, megestrol acetate, melphalan L-PAM, mercaptopurine 6-MP, mesna, methotrexate, methoxsalen, mitomycin C, mitotane, mitoxantrone, nandrolone phenpropionate, nelarabine, nofetumomab, oprelvekin, oxaliplatin, paclitaxel, paclitaxel protein-bound particles, palifermin, pamidronate, panitumumab, pegademase, pegaspargase, pegfilgrastim, peginterferon alfa-2b, pemetrexed disodium, pentostatin, pipobroman, plicamycin, mithramycin, porfimer sodium, procarbazine, quinacrine, rasburicase, rituximab, sargramostim, sorafenib, streptozocin, sunitinib, sunitinib maleate, talc, tamoxifen, temozolomide, teniposide VM-26, testolactone, thalidomide, thioguanine 6-TG, thiotepa, topotecan, topotecan hcl, toremifene, tositumomab, tositumomab/I-131 tositumomab, trastuzumab, tretinoin ATRA, uracil mustard, valrubicin, vinblastine, vincristine, vinorelbine, vorinostat, zoledronate, zoledronic acid and mixtures thereof.

In a pharmaceutical composition aspect of the present invention at least one compound selected from the group consisting of bepridil, lidoflazine, nicardipine, propafenone, rescinnamine, GBR 12909, ellipticine, hexestrol, loxapine, pimozide, acacetin, mometasone furoate or its active 6-β-hydroxy metabolite, ketoconazole and cyclosporin A, and mixtures thereof or their pharmaceutically acceptable salts (preferably, bepridil, nicardipine, propafenone, rescinnamine, ketoconazole, cyclosporine A, loxapine, pimozide, acacetin, mometasone furoate, its active 6β-hydroxy metabolite and mixtures thereof) is combined with at least one additional anticancer compound (agent) in an effective amount in combination with a pharmaceutically acceptable carrier, additive or excipient to treat cancer, or to reduce the likelihood of an occurrence, a recurrence or metastasis of any one or more of the cancers specifically identified in the present application.

The above identified compound(s) may be combined with at least one agent selected from the group consisting of antimetabolites, Ara C, etoposide, doxorubicin, taxol, hydroxyurea, vincristine, cytoxan (cyclophosphamide) or mitomycin C, among numerous others, including topoisomerase I and topoisomerase II inhibitors, such as adriamycin, topotecan, campothecin and irinotecan, other agent such as gemcitabine and agents based upon campothecin and cisplatin for the treatment of cancer, as otherwise described herein. Additional agents which may be combined in pharmaceutical compositions according to the present invention include, for example, adriamycin, anastrozole, arsenic trioxide, asparaginase, azacytidine, BCG Live, bevacizumab, bexarotene capsules, bexarotene gel, bleomycin, bortezombi, busulfan intravenous, busulfan oral, calusterone, campothecin, capecitabine, carboplatin, carmustine, carmustine with polifeprosan 20 implant, celecoxib, cetuximab, chlorambucil, cisplatin, cladribine, clofarabine, cyclophosphamide, cytarabine, cytoxan, cytarabine liposomal, dacarbazine, dactinomycin, actinomycin D, dalteparin sodium, darbepoetin alfa, dasatinib, daunorubicin liposomal, daunorubicin, daunomycin, decitabine, denileukin, denileukin diftitox, dexrazoxane, dexrazoxane, docetaxel, doxorubicin, doxorubicin liposomal, dromostanolone propionate, eculizumab, Elliott's B Solution, epirubicin, epirubicin hcl, epoetin alfa, erlotinib, estramustine, etoposide phosphate, etoposide VP-16, exemestane, fentanyl citrate, filgrastim, floxuridine (intraarterial), fludarabine, fluorouracil 5-FU, fulvestrant, gefitinib, gemcitabine, gemcitabine hcl, gemicitabine, gemtuzumab ozogamicin, goserelin acetate, goserelin acetate, histrelin acetate, hydroxyurea, ibritumomab tiuxetan, idarubicin, ifosfamide, imatinib mesylate, interferon alfa 2a, interferon alfa-2b, irinotecan, lapatinib ditosylate, lenalidomide, letrozole, leucovorin, leuprolide acetate, levamisole, lomustine CCNU, meclorethamine, nitrogen mustard, megestrol acetate, melphalan L-PAM, mercaptopurine 6-MP, mesna, methotrexate, methoxsalen, mitomycin C, mitotane, mitoxantrone, nandrolone phenpropionate, nelarabine, nofetumomab, oprelvekin, oxaliplatin, paclitaxel, paclitaxel protein-bound particles, palifermin, pamidronate, panitumumab, pegademase, pegaspargase, pegfilgrastim, peginterferon alfa-2b, pemetrexed disodium, pentostatin, pipobroman, plicamycin, mithramycin, porfimer sodium, procarbazine, quinacrine, rasburicase, rituximab, sargramostim, sorafenib, streptozocin, sunitinib, sunitinib maleate, talc, tamoxifen, temozolomide, teniposide VM-26, testolactone, thalidomide, thioguanine 6-TG, thiotepa, topotecan, topotecan hcl, toremifene, tositumomab, tositumomab/I-131 tositumomab, trastuzumab, tretinoin ATRA, uracil mustard, valrubicin, vinblastine, vincristine, vinorelbine, vorinostat, zoledronate, zoledronic acid and mixtures thereof.

Preferred pharmaceutical compositions according to the present invention comprise an effective amount of at least one compound selected from the group consisting of bepridil, nicardipine, propafenone, rescinnamine, ketoconazole, cyclosporine A, loxapine, pimozide, acacetin, mometasone furoate, its active 6β-hydroxy metabolite and mixtures thereof, or their pharmaceutically acceptable salts and mixtures thereof (preferably, including at least mometasone furoate, its active 6β-hydroxy metabolite or a pharmaceutically acceptable salt thereof), in combination with one or more anticancer agents as otherwise disclosed herein and in particular, at least one compound selected from the group consisting of anthracyclines (daunorubin, doxorubicin, epirubicin, idarubicin, and valrubicin), the vinca alkaloids (vincristine, vinblastine, vindesine and vinorelbine), taxanes (paclitaxel or taxol, and docetoxel or taxotere), epidopodophyllotoxins (etoposide or VP-16 and tenoposide), nelarabine and imatinib, or a pharmaceutically acceptable salt thereof, among others.

At the first onset of cancer or at the first indication that a patient is at risk for the occurrence or recurrence of cancer, for example because of the isolation and analysis of precancerous cells or other conditions which evidence that a precancerous condition may worsen into a cancer disease state or alternatively, metastasize to other tissue, an effective amount of at least one ABCB1 inhibitor as otherwise described herein is coadministered with at least one anticancer agent as described herein to treat the patient for a time and in a manner which is appropriate for avoiding the cancer or metastasis of the cancer and/or causing the cancer to go into remission or at least to extend the life of the patient. Although the present method may be used quite effectively to treat cancers which are drug resistant and especially those exhibiting multiple drug resistance, the present method is used to treat any cancer in order to reduce the likelihood that a cancer will develop drug resistance during treatment, reduce the likelihood that the cancer will recur and reduce the likelihood that should such cancer recur, that the recurring cancer is drug resistant or will exhibit multiple drug resistance.

Thus, the present compounds and compositions may be used quite effectively to treat cancers, especially those which are drug resistant or exhibit multiple drug resistance and which provide an exceptionally effective treatment modality to reduce the risk of occurrence, recurrence and/or metastasis of a cancer, especially a drug resistant cancer or a cancer which exhibits multiple drug resistance.

By way of history, as discussed, the identification of ABCB1 inhibitors and pharmaceutical compositions and methods of treatment of cancer according to the present invention emerged from the screening of a chemical library of compounds to determine their potential for ABCB1 inhibition. Pursuant to this methodology, which is applicable for identifying inhibitors of other ABC transporter proteins, we now turn to a description of the method which is useful to identify inhibitors of transporter proteins, which may ultimately be shown to assist in the treatment of certain cancers, especially drug resistant cancers.

### Methods of Identifying Inhibitors of ABCB1, ABCG2 and ABCC1 Transporter Proteins

The present method also relates to methods for identifying inhibitors of ABCB1, ABCG2 and ABCC1 transporter proteins utilizing high throughput flow cytometry. The present methods allow high throughput screen of hundred or thousands of compounds within a relatively short period of time in order to determine their ability to modulate (primarily inhibition, which can be direct or indirect, including allosteric inhibition). Described are the methods, experiments and systems which have been provided to identify the appropriate compounds having activity in modulating ABCB1, ABCG2 and/or ABCC1 transporter proteins. Inhibitors are to be identified to determine what, if any, activity these compounds may have in being used in the treatment of disease.

The methods of the present invention utilize a system which provides an assay for determining the impact of any number of compounds of unknown activity on ABCB1, ABCG2 and ABCC1 transporter proteins to determine inhibitors which may prove potentially useful in cancer therapy.

In particular, the following methods are provided pursuant to the present invention:

In one embodiment, the present invention is directed to a method to measure the ability of test compounds to inhibit the function of ABCB1 and ABCG2 transporters in a single assay, comprising the steps of:

labeling Jurkat-DNR cells (for measuring modulation, especially inhibition of ABCB1 transporter proteins) with FarRed DDAO CellTrace SE (which is available from a number of suppliers, including Invitrogen);

washing the Jurkat-DNR cells;

combining the Jurkat-DNR cells with unlabeled IgMXP3 cells (for measuring inhibition/modulation of ABCG2) in an assay buffer to form a cell suspension, allowing the label to bind covalently to amine groups;

adding JC1 (fluorescent substrate) solution at an effective final concentration (preferably less than 2% to the cell suspension);

subsequently dispensing cells from the cell suspension into well plates;

adding test and control compounds to the cell suspension in the wells;

incubating for the cell suspension in the wells; and

delivering the cell suspension from the wells to a flow cytometer to determine pump activity and the inhibitory activity of the compounds tested.

In the present method described above, more particularly, the Jurkat-DNR cells (ABCB1 modulation/inhibition) are labeled preferably with approximately 0.5 ng/ml FarRed DDAO CellTrace SE (available from Invitrogen) for approximately 15 min or so at room temperature. The washing of said Jurkat-DNR cells is done by centrifugation and is performed at least twice. The flow cytometer which is used is operated to detect the label at red fluorescence emission wavelengths of 665±10 nm upon excitation at 635 nm. In the above method the cell suspension is fed to the flow cytometer which has a final in-well concentration of about 3×10⁶ cell/ml at a 1:1 ratio of the two cell types to analyze, with approximately 1,000 cells of each cell type from each well being delivered to the flow cytometer per sample when sampling at 40 wells/min. The JC1 (substrate) solution is added at a final concentration of approximately 0.2% to the cell suspension. In the present method, the cell suspension preferably is dispensed into 384 well plates at approximately 100 ul/well and the test and control compounds are added to the wells at approximately 1 ul/well and incubated for about 15 minutes or so at room temperature.

In another embodiment of identifying inhibitors of ABC transporter proteins, the present invention is directed to a method to measure the ability of test compounds to inhibit the function of ABCB1 and ABCC1 transporters, comprising the steps of:

labeling CCRF-Adr cells (which measures ABCB1 modulation/inhibition) with an effective amount of FarRed DDAO Cell Trace SE;

washing said CCRF-Adr cells;

combining said CCRF-Adr cells with unlabeled SupT1-Vincristin cells (which measure ABCC1 modulation/inhibition) in an assay buffer to form a cell suspension;

adding an effective amount of labeled CaAM (fluorescent substrate) to the cell suspension;

subsequently incubating the cell suspension;

distributing said cell suspension into wells of a well plate;

adding test and control compounds to the wells;

subsequently incubating the cell suspension in the wells; and

delivering the cell suspension from the wells to a flow cytometer to determine pump activity and activity of the compounds tested.

In the above embodiment, the method preferably comprises the washing (multiple steps) of said CCRF-Adr cells and the CaAM is added to the cell suspension to an effective final concentration, e.g., preferably about 250 nM. and the incubating of the cell suspension after the addition of CaAM thereto preferably is carried out for about 10-15 minutes at 37° C. The cells suspension is introduced into the wells at approximately 100 ul/well and the test and control compounds preferably are added in a volume amount of approximately 1 ul/well. The incubating of the cell suspensions in the wells after the adding of the test and control compounds is carried out for approximately 15 minutes at room temperature. The flow cytometer is preferably the Hypercyt system.

In yet another embodiment, the present invention relates to a method used to find inhibitors of ATP binding cassette (ABC) transporter protein pumps (ABCB1, ABCC1 and ABCG2) contemporaneously, comprising:

configuring well microplates (preferably 384) with control wells (preferably 64): configuring columns (e.g., 1, 2, 23 and 24) for negative unblocked transporter activity controls and positive blocking (transporter inhibition) controls wherein JC1 and CaAM (fluorescent substrates) serve as markers for active transport function by measurement of retained fluorescence per cell;

screening the three transporter protein pumps against test compound libraries by providing (constructing) two overlapping duplex transporter assays, the first of said duplex transporter assays using JC1 as substrate to quantify ABCB1 and ABCG2 activity, and the second of said duplex transporter assays using CaAM as substrate to quantify ABCB1 and ABCC1 activity;

using effective amounts of Nicardipine and Verapamyl as positive controls (each preferably at about 50 μM final concentration) in said first JC1 and second CaAM duplex assays, respectively; and

adding test compounds, stored in solution (e.g. DMSO) stocks to the remaining wells (e.g., 32) at final effective concentrations of up to ˜5 μM, and

determining the activity of the compounds tested.

Thus, in the present invention, two separate duplex assays were constructed: one in which ABCB1 and ABCG2 transporters were evaluated in parallel using fluorescent JC1 (fluorescent probe) as substrate and the other in which ABCB1 and ABCC1 transporters were evaluated in parallel using fluorescent probe calcein-AM (CaAM) as substrate. ABCB1-expressing cells were color coded to allow their distinction from cells expressing the alternate transporter. The assays were validated in a screen of the Prestwick Chemical library, a collection of 880 off-patent small organic molecule drugs and alkaloids. A number of ABCB1 inhibitors were identified including selective inhibitors of the ABCC1 transporter (loxapine, pimozide or acacetin), and the activity of each was confirmed in follow up chemosensitivity shift and reversal studies.

### Particular Features of Assays and Methods for Identifying Inhibitors

### Multiplex Assay Design

The screen has been formatted for 384 well plates using a total of 100 μl in each well. 384 well microplates are configured with 64 control wells: columns 1, 2, 23 and 24 for negative unblocked transporter activity controls (1% DMSO) and positive blocking (transporter inhibition) controls. JC1 and CaAM serve as markers for active transport function simply by measurement of retained fluorescence per cell. JC1 was determined to be a substrate for both ABCB1 and ABCG2 transporters, but a poor substrate for the ABCC1 transporter. CaAM was a good substrate for ABCB1 and ABCC1 but not ABCG2. In order to efficiently screen the three pumps against test compound libraries, a pair of overlapping duplex transporter assays was constructed, one using JC1 to quantify ABCB1 and ABCG2 activity, the other using CaAM to quantify ABCB1 and ABCC1 activity. Nicardipine and Verapamyl are used as positive controls (each at 50 μM final concentration) in JC1 and CaAM duplex assays, respectively. Test compounds, stored as DMSO stocks, were added to the remaining 320 wells. Test compounds were typically screened at final concentrations of up to ˜5 μM.

### JC1 Duplex Assay.

The JC1 duplex assay measured the ability of test compounds to inhibit the function of ABCB1 and ABCG2 transporters. Jurkat-DNR cells (ABCB1) were labeled with 0.5 ng/ml FarRed DDAO CellTrace SE (Invitrogen) for 15 min at room temperature, washed twice by centrifugation, then combined with unlabeled IgMXP3 cells (ABCG2) in the assay buffer. The label binds covalently to amine groups in cells and is detected at red fluorescence emission wavelengths of 665±10 nm upon excitation at 635 nm. Final in-well concentration of cells was 3×106 cell/ml at a 1:1 ratio of the two cell types. This resulted in analysis of ˜1,000 cells of each cell type from each well when sampling at 40 wells/min (aspirated sample volume ˜2f-l1). JC1 solution at a final concentration of 0.2% was added to the cell suspension after which cells were dispensed into 384 well plates at 100 ul/well, test and control compounds were added to wells (1 ul/well). After 15 min incubation at room temperature, cells were evaluated in the flow cytometer to determine pump activity.

### Calcein AM(CaAM) Duplex Assay.

The CaAM duplex assay measured the ability of test compounds to inhibit the function of ABCB1 and ABCC1 transporters. Transporter inhibition is measured by the accumulation of the fluorescent free calcein inside cells. It is very sensitive method due to the enzymatic enhancement of the dye trapping process. For each 384 well plate, CCRF-Adr cells (ABCB1) were labeled with FarRed DDAO Cell Trace SE, washed twice and combined with unlabeled SupTI-Vincristin cells (ABCC1) exactly as described for the two cell lines in the JC1 Duplex assay. CaAM was then added to the cell suspension to a final concentration of 250 nM, incubated 10 min at 37° C. and distributed into the 384 well plate at 100 ul/well. Test and control compounds were added to wells (1 ul/well), After 15 min incubation at room temperature, cells were evaluated in the Hypercyt@ system to determine pump activity.

### High Throughput Flow Cytometry Analysis

The HyperCyt® system was used for aspiration of samples from 384 well microplates and delivery to a Cyan flow cytometer (Dako Cytomation) for analysis. As the sampling probe of the HyperCyt autosampler moves from one well to the next of a multiwell microplate, a peristaltic pump sequentially aspirates sample particle suspensions from each well. Between wells, the continuously running pump draws a bubble of air into the sample line. This results in the generation of a tandem series of bubble-separated samples for delivery to the flow cytometer. Immediately after data acquisition by the flow cytometer, specialized software (IDLeQuery) was used to analyze the data file. The program automatically detects the time-resolved data clusters (each representing data from a single well), and analyzes each to determine the median channel fluorescence (MCF) of JCI or CaAM fluorescence. These data were automatically exported to a Microsoft Excel spreadsheet template that calculates the assay quality control Z′ factor and transporter inhibition percent for each well.

### Quantitative Assay Evaluation

Test compound % inhibition of efflux pump activity at each point was calculated as 100×[1−(MFI_PC_MFI_Test)/(MFI_PC−MFI_NC)] in which MFI Test, MFI_PC and MFI_NC represent the median fluorescence intensity (MFI) of cells in wells containing test compound, the average MFI of cells in positive control wells (maximum fluorescence intensity) and the average MFI of cells in negative control wells (minimum fluorescence intensity), respectively. Zprime, signal to noise (S/N) and signal to background (S/B) statistics were calculated as described in Zhang et al (J Biomol Screen 4:67, 1999). Each 384 well plate contained 32 positive control wells and 32 negative control wells that were used for quality statistic calculations.

### Testing for Inhibition of Multiple Drug Resistant Forms of Cancer

The inventors developed a chemo-resistant T-ALL cell line that could be tested for ABCB1-reversal agents using JC-1, a cationic dye that exhibits differential fluorescence based on its intra-cellular concentrations. An initial screen identified 19 target compounds and, based on a published record of safe use in humans, 11 compounds were investigated for further analysis. On the basis of an unfavorable in vitro toxicity profile, the inventors excluded four drugs, leaving seven compounds for further analysis. Later, three additional compounds were added to the list. These seven drugs have diverse structural and functional classifications, and include cation channel blocking agents, an angiotensin converting enzyme (ACE) inhibitor, an imidazole, and an immunosuppressant. Through HTS for ABCB1 reversal agents, the inventors also identified mometasone furoate as a drug that works as an inhibitor or efflux blocker of ABCB1 transporter protein.

### Materials and Methods

### Reagents, Monoclonal Antibodies and Cells

Daunorubicin (DNR), prednisolone (PRED), and L-asparaginase (L-asp) were purchased from Sigma-Aldrich (St. Louis, Mo.). Vincristine (VCR) was obtained from Faulding Pharmaceutical Company (Paramus, N.J.). Phycoerythrin-conjugated (PE) mAbs against ABCB1 (USBiological, Swampscott, Mass.) and a PE-conjugated IgG₂ₐ isotype control (Becton Dickinson, San Jose, Calif.) were used in accordance with the manufacturer's recommendations. Jurkat cells were purchased from American Tissue Culture Corporation (Manassas, Va.). As previously described,¹⁰ cells were grown in RPMI 1640 medium (Gibco BRL, Grand Island, N.Y.) with 100 units/mL penicillin-streptomycin (Gibco), 0.5 μL/L Ciprofloxacin (Bayer Pharmaceuticals, Berkeley, Calif.), and 10% heat-inactivated fetal bovine serum (FBS). Cells were incubated at 37° C. in a humidified atmosphere of air/5% CO₂.

### Microarray Pre-Processing and Analysis

Total RNA was extracted using an RNeasy Mini Kit (Qiagen). RNA processing and hybridization to the Human Genome U133 Plus 2.0 oligonucleotide microarray (Affymetrix, Santa Clara, Calif.) were performed according to the manufacturer's protocol. Signal intensity was scaled to 500 and collected by Affymetrix GeneChip Operating Software (GCOS). Probeset intensities were normalized with the Robust Multi-Array Average (RMA) method⁴ in GeneSpring 7.1 (Agilent Technologies, Palo Alto, Calif.). Fold-change comparisons between parental and resistant cell lines were made using GeneSpring 7.1 (Agilent).

### Flow Cytometric Analysis of ABCB1 Cell Surface Expression

Daunorubicin-resistant and wild-type Jurkat cells were centrifuged twice at 800 rpm (4° C.) for 3 min and re-suspended in 1×PBS. The cells were then incubated on ice under dark conditions for 30 min with either the PE-conjugated ABCB1 or the IgG₂ₐ isotype control. After two additional rounds of centrifugation, the cells were analyzed for surface protein expression using a FACscan flow cytometer (Becton Dickinson).

### JC-1 Assay and Flow Cytometry

A recently developed flow cytometric assay evaluated the P-gp function by measuring the accumulation of J-aggregate forming lipophilic cation 5,5′,6,6′-tetrachloro-1,1′,3,3′-tetraethylbenzimidazolcarbocyanine iodide (JC-1) in the absence or the presence of a P-gp inhibitor. Due to the stacking in a liquid crystal form, the fluorescence emission wavelength of this probe depends on its concentration. When the JC-1 monomers are excited at 488 mm, the emission spectrum reaches its maximum at 537 nm (green fluorescence). Beyond a critical concentration, JC-1 aggregates are formed and, in addition to green fluorescence, display a red fluorescence at 597 nm. While sensitive cells display both green and fluorescence, resistant cells display only green fluorescence. At least 10,000 events were counted (FACscan) and analyzed with IDLeQuery software.⁵

### Prestwick Chemical Library

The Prestwick chemical library is a collection of 880 off-patent small organic molecules, of which approximately 85% of the compounds have been marketed as drugs or biologically active agents. Prestwick Chemical Library compounds were provided as DMSO stock solutions and were diluted 1:50 in assay dilution buffer (ADB; 110 mM NaCl, 30 mM HEPES, 10 mM KCl, 1 mM MgCl₂, 10 mM glucose, and 0.1% bovine serum albumin) to attain DMSO concentrations of 2% prior to addition to wells. Final DMSO concentration in the assay was 1%.

### Flow Cytometric Detection of ABCB1 Reversal (HyperCyt® Assay)

The quantification of ligand binding, surface antigen expression, and characterization of cellular immunophenotypic features has been previously reported for the HyperCyt assay.¹ The HTS response data were measured in the FL1 green fluorescence emission channel (530-540 nm), and the FL3 red fluorescence emission channel (>650 nm) was used for detection of Cytoplex L9 or L10 beads (Duke Scientific, Palo Alto, Calif.). The beads were used as an internal control to facilitate the proper registration of flow cytometry data with source wells. Jurkat cells grown in the presence of 20 nM DNR were centrifuged and resuspended in 1×JC-1 buffer. 100 μl of this cell suspension was added to a 96-well PCR plate (GeneMate) containing 80 different Prestwick compounds at a final concentration of 5 μM. The first column of eight wells contained no compounds (negative control) and included Cytoplex L9 or L10 beads to delineate the beginning of a row and the last column of eight wells contained 10 μM cyclosporine A (positive control). Secondary screening of “hits” was performed in 384-well format by the same protocol except serial dilutions of the compound of interest replaced the test compounds. From these dose response data, an EC50 was determined using Prism (GraphPad Software, San Diego, Calif.).

### Determination of Chemᵣₑᵥₑᵣₛₐₗ₅₀ and TD₅₀ Concentrations

To quantify the effects of ABCB1-reversal agents in conferring sensitivity in DNR resistant cells, the inventors determined the ABCB1-reversal agent drug concentrations that resulted in a 50% decrease of cell viability in 100,000 Jurkat cells/mL maintained in a fixed concentration of 100 nM DNR (Chemᵣₑᵥₑᵣₛₐₗ₅₀). To enable this analysis, a 3-log range of each “hit” compound was added in 2 mL aliquots to a 6 well plate and, over a range of 7 days, total cell number and viability was measured using a hemacytometer and trypan blue staining. Fresh media containing 100 nM DNR was added over the course of the experiment to maintain the cell concentration ≧100,000 cells/mL. Using a similar approach, the inventors measured the direct toxic effects of the ABCB1-reversal agents that resulted in cell death in DNR-resistant Jurkat cells maintained in media alone (Toxic Dose₅₀; hereafter, TD₅₀). Results were compared to the survival of parental cells in the presence of DNR (the positive control resulted in 100% cells death) as well as the survival of drug resistant cells in the presence of chemotherapeutic drug (the negative control yielded 100% cell viability). The differences (if any) between the Chemᵣₑᵥₑᵣₛₐₗ₅₀ and TD₅₀ gave an approximation of an in vitro “therapeutic index” for each compound.

### Statistical Analysis

To quantify intracellular variations in JC-1 fluorescence, time-resolved HTS data were analyzed using IDLeQuery; this program detects data clusters and analyzes each to determine the Z scores for fluorescence. To calculate Z scores for each HTS data set, the following equation was employed: Z′=1−[(3×NC_{SD})+(3×PC_{SD})]/(NC_{AVG}−PC_{AVG}). In this equation, NC_{SD} describes the standard deviation of the mean fluorescence intensity (MFI) of cells in negative control wells, PC_{SD} describes the standard deviation of the MFI of cells in the positive control wells; NC_{AVG} and PC_{AVG} describe the negative and positive average MFIs of cells in control wells, respectively. Because each sample consists of 2 μl taken from a 15 μl volume in each well, the inventors routinely sampled and analyzed each plate twice and averaged the results

Ligand competition curves were fit by nonlinear least-squares regression using a 1-site competition model with Prism® software (GraphPad Software, Inc., San Diego, Calif.) to determine the concentration of added competitor that inhibited fluorescent ligand binding by 50% ([IC₅₀]). The inventors used the Spearman's rank correlation coefficient (Rs) to study the correlation between different parameters. After classifying the results in a frequency table, a Chi-square (c²) test was performed to study the relationship between the different categories.

### Results

### Development of an ABCB1-Upregulated T-ALL Cell Line

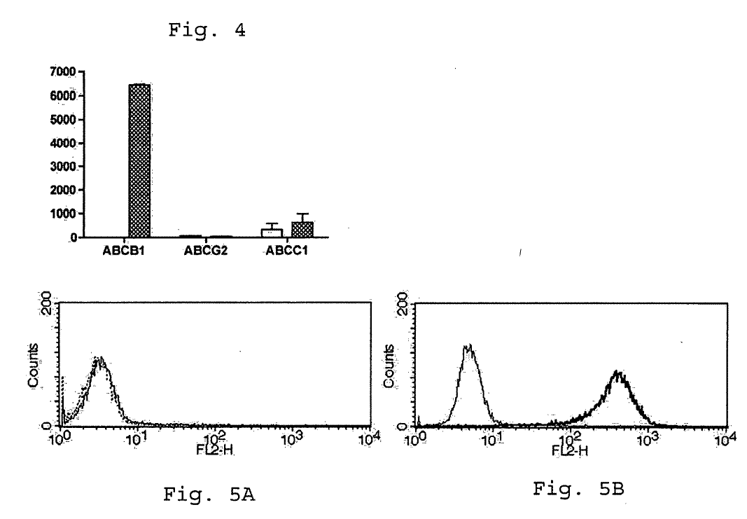

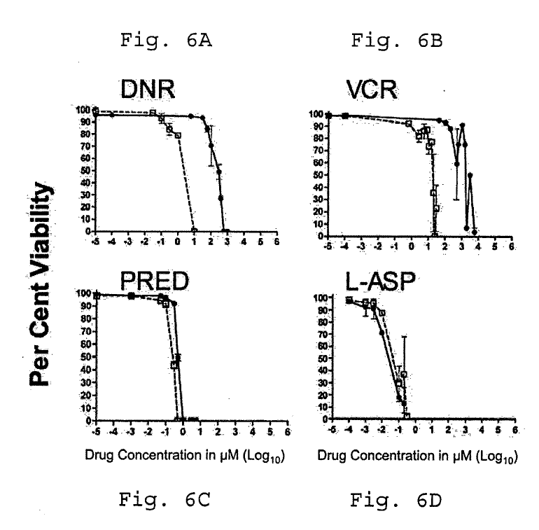

ABCB1 acts as a molecular pump that is capable of lowering intracellular concentrations of substrate xenobiotics, such as DNR, VCR, and PRED and a number of other chemotherapeutic agents. To investigate whether JC-1 might be used to quantify the effectiveness of ABCB1 efflux inhibitors, the inventors first developed a T-ALL cell line with acquired resistance to DNR, a well-known ABCB1 transport substrate.³ To confirm that DNR-induced multi-drug resistance was conferred by inducible up-regulation of the ABCB1 gene, the inventors measured relative changes in ABCB1 mRNA expression between the Jurkat wild type and DNR-resistant cell lines. The inventors observed that ABCB1 mRNA was selectively up-regulated approximately 600-fold in the resistant cell line (FIG. 4). When comparing fold up-regulation for other ABC transporters, to specifically include ABCC1 (multiple resistant protein, MRP) and ABCG2 (breast cancer-related protein; BCRP), the inventors also found that mRNA levels remained unchanged (less than 1.5-fold difference) between the resistant and wild-type Jurkat cell lines. To determine whether up-regulation of ABCB1 mRNA resulted in increased surface expression of the ABCB1 protein, flow cytometry was used to test for ABCB1 protein expression in the Jurkat cells lines. It was found that in comparison to chemo-sensitive cells (FIGS. 5A and 5B), ABCB1 expression was up-regulated by more than 100-fold on resistant cells. The inventors next tested Jurkat DNR resistant cells for acquired resistance to VCR, PRED and L-ASP, drugs that are commonly used to induce remission from T-ALL.⁶ It was found that Jurkat cells having acquired resistance to DNR also showed an approximately 70 fold increased resistance to VCR (IC₅₀ 0.8 vs 2.5 μM), a 3 fold increased resistance PRED (IC₅₀-0.5 vs. 0 μM), but no increased resistance to L-ASP (FIGS. 6A-6D). These data show that the DNR-resistant Jurkat cell line demonstrates resistance to chemotherapeutic agents having a diverse array of structure and class distinctions.

### Adaptation of the HyperCyt® Assay to Identify an ABCB1-Reversal ‘Signature’

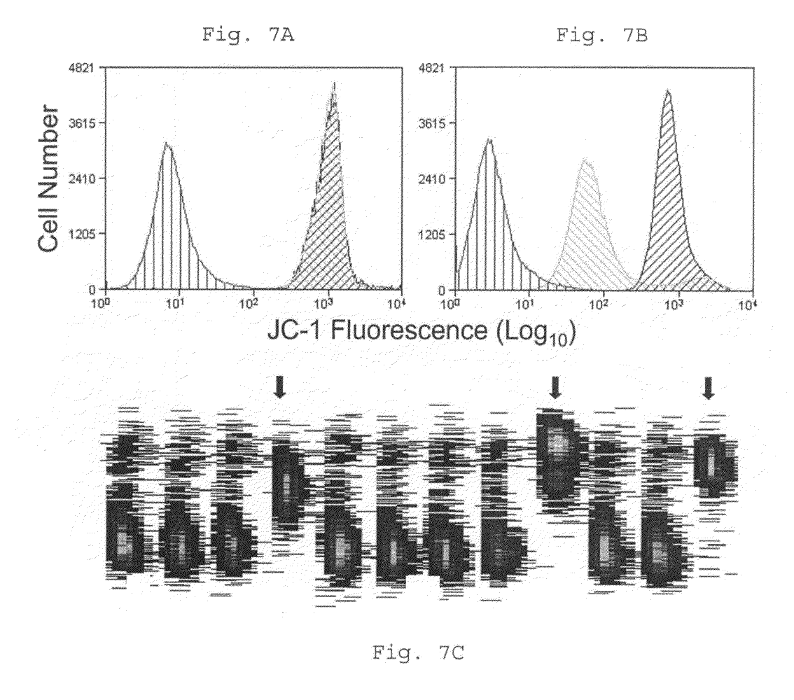

Having established that chemo-resistant Jurkat cells express upregulate ABCB1 in the context of cytotoxic pressure, the inventors next assessed whether cyclosporine A (CSA), a potent drug transport efflux inhibitor, might alter intracellular JC-1 concentrations in a flow cytometric quantification. In comparison to unblocked chemo-resistant cells, CSA inhibited the efflux of JC-1 from DNR-resistant Jurkat cells (FIGS. 7A and 7B). Based on the findings of Legrand et al.² that JC-1 may be used to measure ABCB1 transport, the HyperCyt system was adapted to distinguish ‘chemical hits’ in Jurkat cells showing a differential in intracellular JC-1 concentrations (FIG. 7C). It was found that CSA and other ABCB1 drug efflux inhibitors could be identified by shifts in intracellular JC-1 concentrations and consequent fluorescence levels. This approach also allowed for the rapid screen of many compounds, thus avoiding the necessity of maintaining cells in culture for long-term periods, and likely also contributed to the high reliability of the assay. The Z′ statistics for the plate measurements (analyzed in duplicate) were 0.57±0.25 (mean±SD) for FL2 and 0.78±0.13 for FL1, indicating a high level of reproducibility. Based on the concentration of the ABCB1 reversal agent, shifts in intracellular JC-1 fluorescence show a continuum of effect, allowing the exclusion of compounds having low-level signals.

Identification of Prestwick Compounds that Block ABCB1-Mediated JC-1 Efflux

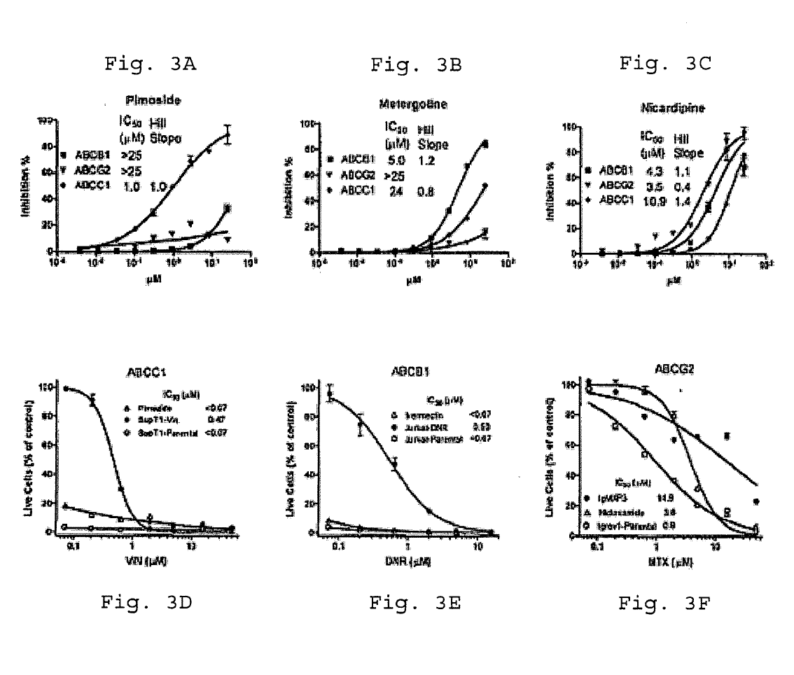

From 880 potential targets within the Prestwick Chemical Library, the inventors identified 19 compounds (2%) as inhibiting ABCB1-mediated drug efflux (FIG. 3). Because it was desired to identify agents that might enhance chemotherapeutics, the inventors filtered from their analysis compounds that could not be taken internally, had unproven benefit, or had been previously reported to have unacceptable toxicities in humans. Using these criteria, eight additional drugs (antimycin, thiocolchicoside, methiothenin maleate, fusidic acid, rotenone, ciclopirox ethanolamine, syrosingopine and avermectin) the inventors excluded from the initial list of 19 compounds, leaving 11 candidate compounds available for further investigation as chemo-sensitizing agents. Three additional candidates were uncovered in separate experiments. These are loxapine, pimozide and acacetin.

The inventors next performed IC₅₀ analyses for each of 11 drugs to determine the relative drug efflux activity over the concentration range 0.1 nM to 1 μM. Of the 11 compounds identified for IC₅₀ analyses, 4 drugs are cation channel blockers, including the calcium channel blockers bepridil, lidoflazine and nicardipine, and the sodium channel blocker, propafenone (FIG. 8A). For the calcium channel blockers, the IC₅₀s were 0.76±0.18 μM for nicardipine, 5.4±0.55 μM for lidoflazine and 7.4±0.95 μM for bepridil. Propafenone had the highest IC₅₀, which was measured to be 19.3±2.1 μM. The screen identified three drugs having physiologic effects at intra-nuclear sites of activity, including one topoisomerase inhibitor, ellipticine, and two steroids, mometasone furoate and hexestrol (FIG. 8B). Ellipticine was determined to have an IC₅₀ of 0.99±0.014 μM. The steroid derivatives mometasone furoate and hexestrol had IC₅₀s ranging from 0.74±0.13 to 8.3±3.2 μM, respectively. The remaining four drugs in the screen belong to drug classes having diverse functions and structures (FIG. 8C). These ABCB1 reversal agents (and their respective IC₅₀ concentrations) include rescinnamin (1.53±0.5 μM), an angiotensin converting enzyme inhibitor; GBR 12909 (4.6±0.28 μM), an inhibitor of dopamine uptake; ketaconazole (2.18±0.45 μM), an antifungal; and cyclosporine A (0.31±0.087 μM), a potent immunosuppressant and inhibitor of DNA binding activity. Altogether, these drugs showed a 62-fold range in IC₅₀ concentrations in the DNR-resistant Jurkat cell line. With the exception of nicardipine, the cation-channel blocking agents had higher IC₅₀s then most other agents. Conversely, drugs with intra-nuclear sites of activity had lower IC₅₀ concentrations, suggesting that sub-cellular site of activity might influence each drug's anticipated efficacy and toxicity profile.

### Secondary Screen for Cellular Chemosensitization and Toxicity

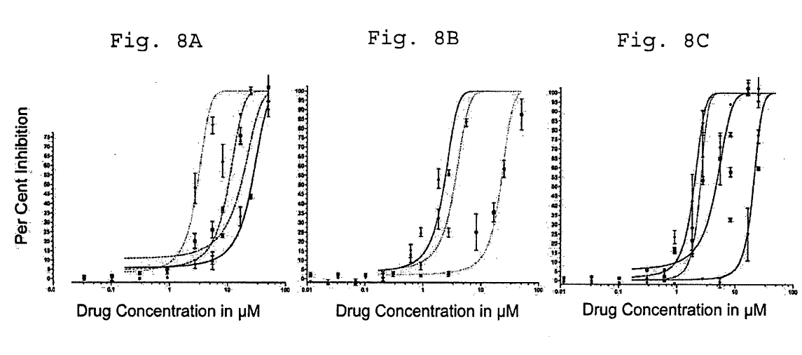

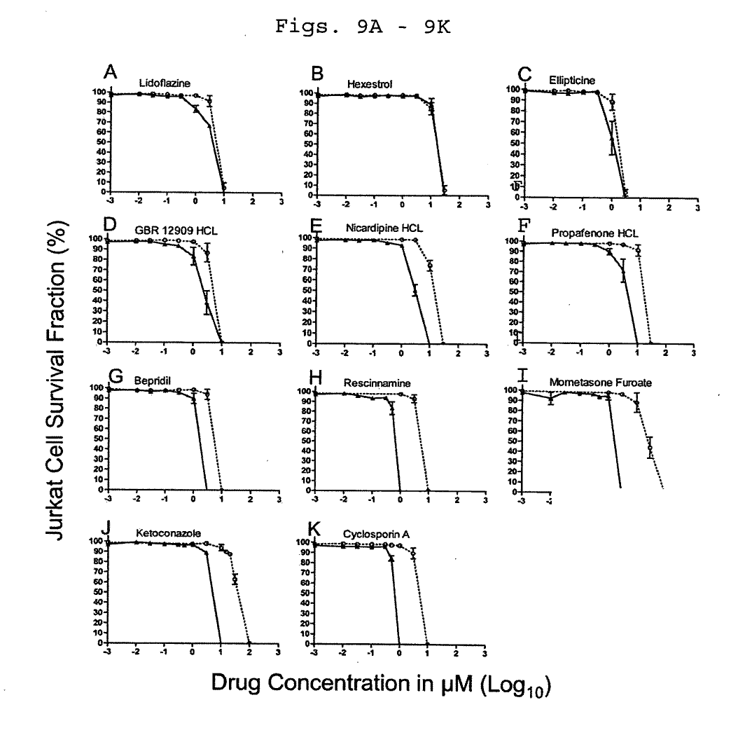

To verify whether the 11 compounds may have cellular effects that potentiate the effects of DNR, two functional assays (FIGS. 9A-9K) were carried out. In the first, the inventors tested each compound's potential to sensitize the DNR-resistant Jurkat cells to the cytotoxic effects of DNR (Chemᵣₑᵥₑᵣₛₐₗ₅₀), and in the second the inventors assessed each compound's direct in vitro toxicity (TD₅₀). In the Chemᵣₑᵥₑᵣₛₐₗ₅₀ assay, Jurkat viability was assessed against a 5-log range of efflux inhibitor compounds in a fixed concentration of DNR and, for each of the 11 drugs, dose-dependent decreases in cell viability were observed. Within the cation channel blocking class of compounds, the inventors again observed a range of drug concentrations that could reverse DNR-resistant Jurkat cells line to chemo-sensitivity. For the calcium channel blocking agents, the chemᵣₑᵥₑᵣₛₐₗ₅₀s were 3.3±2.3, 3.1±1.7 and 2.6±1.1 μM, respectively for bepridil, lidoflazine and nicardipine, and for the sodium channel blocking agent propafenone, the chemᵣₑᵥₑᵣₛₐₗ₅₀ was 4.9±1.3 μM. For the steroid derivatives mometasone and hexestrol, the chemᵣₑᵥₑᵣₛₐₗ₅₀s were measured to be 1.4±0.28 and 12.8±3.3 μM, respectively; the topoisomerase inhibitor ellipiticine had a chemᵣₑᵥₑᵣₛₐₗ₅₀ of 1.8±0.92 μM. In the remaining drug classes, the following respective chemᵣₑᵥₑᵣₛₐₗ₅₀s were identified: rescinnamine, 1.0±0.5; GBR 12909, 2.1±0.01; ketoconazole, 3.7±0.99; and cyclosporine, 0.63±0.01 μM. Overall, the chemᵣₑᵥₑᵣₛₐₗ₅₀s for the 11 lead compounds ranged 20-fold, from cyclosporine having the lowest chemᵣₑᵥₑᵣₛₐₗ₅₀ value, to hexestrol having the highest for the identified ABCB1 reversal agents.

Because ABCB1-reversal agents can have significant toxicities, the inventors tested a 5-log range of drug concentrations for each hit compound to determine the TD₅₀. Four drugs with indistinguishable chemᵣₑᵥₑᵣₛₐₗ₅₀ and TD₅₀ curves were eliminated from consideration. In addition, drugs having a TD₅₀ below its reversal effect or achievable serum concentration would be likely to have a toxicity profile unsuitable for ABCB1 reversal in human use. For the cation channel blocking agents, the TD₅₀s were 24.54±27.2, 9.7±7.6, and 19.4±5.0 μM for bepridil, lidoflazine and nicardipine, and 27.5±5.0 μM for propafenone, respectively. For the steroid derivative hexestrol and mometasone, the TD₅₀s were 10.6±6.4 and 24.8±8.0 μM, respectively, and for ellipticine, the TD₅₀ was 2.7±1.56 μM. Among the remaining compounds, the TD₅₀s were 10.00±6.4 μM for rescinnamine, 5.2±0.14 μM for GBR 12909, 37.9±2.1 μM for ketoconazole, and 3.4±2.34 μM for cyclosporine. Overall, an 11-fold range of TD₅₀s was observed in this set of compounds, the lowest observed in cyclosporine, and the highest measured in ketoconazole.

Comparative Elimination of Drugs Having an Unfavorable In Vitro Therapeutic Index

In this comparative analysis, a goal was to exclude from further consideration compounds having similar or narrowly differing chemᵣₑᵥₑᵣₛₐₗ₅₀ and TD₅₀ concentrations. As shown in FIG. 4, four drugs (lidoflazine, hexestrol, ellipticine and GBR12909) had essentially no differences between their TD₅₀ and Chemᵣₑᵥₑᵣₛₐₗ₅₀ concentrations. Thus, one could not distinguish whether toxicity or efficacy was promoting the inhibitory chemoreversal effects. Accordingly, these four agents were eliminated from further consideration, leaving seven for further investigation (Table 1).

In comparison to the previously described chemo-sensitizing agents that were identified in this screen, the Chemᵣₑᵥ₅₀ curves for rescinnamine and mometasone furoate were most similar to those generated for ketoconazole and cyclosporine. For these compounds, at least a 10-fold difference in drug concentration separated reversal effect from toxic effect. Among the cation channel blocking drugs, nicardipine and propafenone have relatively narrow reversal and toxicity profiles, each being less than 10-fold, suggesting a more restrictive therapeutic index. While correlations between IC₅₀, Chemᵣₑᵥₑᵣₛₐₗ₅₀ and TD₅₀s may help to definitively exclude specific compounds from further analysis, these tests do not replace the need to perform additional studies, to include controlled clinical trials to reliably test drug combinations for safety and efficacy.

Discussion

To improve outcome for cancer patients, there is a critical need to identify new targets for pharmaceutical intervention. The ability of ABCB1 inhibitors to compete with cytotoxic drugs for outward transport has been demonstrated for numerous compounds, which have been collectively termed MDR modulators, efflux inhibitors or chemosensitizing agents. Using a JC-1 adapted, HyperCyt HTS, the inventors evaluated 880 off-patent compounds in the Prestwick Chemical Library for drugs that inhibit ABCB1-mediated efflux. Initially 19 compounds were identified as having potential as chemosensitizing agents. After excluding seven drugs that had no record for safe internal use in humans, and through additional pre-clinical testing, four additional candidates appearing to have unfavorable in vitro therapeutic indices were also removed from consideration, allowing the inventors to further constrain the list of lead candidates to seven compounds. Because ketoconazole, bepridil, nicardipine, and cyclosporine have been previously used as ABCB1-reversal agents in the clinical setting for cancer control, the inventors further constrained their list of lead compounds to three agents. Interestingly, of the remaining three compounds, only mometasone furoate appears to be a novel ABCB1-reversal agent to DNR-mediated drug resistance. The favorable in vitro physiologic profile for this drug merits further investigation for its role as an ABCB1 reversal agent. In addition, the approach indicates that HTS of the Prestwick Chemical Library might be a fruitful approach to identifying lead candidates for novel clinical indications.

Chemical libraries that are comprised of established drugs can be used to screen compounds for new uses, purposes and indications. Using a novel approach to HST, the inventors modified the JC-1 efflux assay for implementation in HyperCyt¹ and, within the Prestwick Chemical Library, measured ABCB1-mediated efflux. The high frequency of “hits”, 2.15% of the library (19 of 880 compounds), is much higher than expected from HTS of random chemical structures. The inventors' experience screening the NIH small molecule repository has typically found less than one active “hit” per 1000 compounds (Sklar and Edwards, unpublished observations). These observations appear to be similar to the experiences of others, indicating that biologically active chemicals (drugs) have a high tendency to interact with multiple molecular targets.

Traditional ABCB1 substrates include anthracyclines (DNR), vinca alkaloids (VCR), taxanes, epidopodophyllotoxins and imatinib. ABCB1 inhibitors, including first through fourth generation compounds, have a medium to low molecular weight, are lipophilic, have two planar aromatic rings, and enter cells by passive diffusion. Mometasone furoate is not an exception. Although these first through fourth generation compounds have been shown to have ABCB1 inhibitory effects in vitro, their results in clinical trials have been mixed. These mixed results have been attributed to the compounds having low bio-availability, unexpected secondary physiological effects, and unanticipated drug-drug interactions. Screening libraries of known drugs might circumvent some of these pitfalls, as most lead compounds will have established drug-dosing specifications and well-established toxicities when used to treat other diseases. The inventors' approach may also expedite the introduction of lead compounds into clinical trials, and avoid the labor-intensive traditional process of optimizing a lead compound over several structural generations.

Mometasone furoate is a potent corticosteroid that clearly inhibits ABCB1 efflux. Interestingly, corticosteroids have tumoricidal effects in leukemias,⁶ leading to the possibility that, in selected cases, their activity may concurrently enhance the effects of other ABCB1 chemotherapy substrates while also providing direct anti-tumor effects.

Tables 2 and 3 disclose some examples of these combinations and examples of types of cancers that they could treat. One embodiment comprises the combination of Mometasone Furoate and/or its 6-beta-hydroxy (active) metabolite (more soluble, better for iv formulations) with Nelerabine, whereby this combination lowers the toxicity of Nelarabine to the subject in treatment. The combinations of tables 2 and 3 are examples to illustrate this disclosure and are not meant to limit the invention in any way. It will be apparent to those skilled in the art that various modifications and variations can be made in the methods and applications of the present disclosure for treatment of cancer or other proliferative diseases. Because Mometasone Furoate is primarily metabolized by cytochrome P450 3A4, care should be exercised when combining this drug with 3A4 inhibitors [e.g., atazanavir, clarithromycin, indinavir, itraconazole, ketoconazole, nefazodone, nelfinavir, ritonavir, saquinavir, telithromycin]. Imatinib has been listed as CYP 3A4 inhibitor. In a preferred embodiment, 6-beta-hydroxy Mometasone Furoate (a product of 3A4 metabolism) is combined with Imatinib.

TABLE 2
Hematological Malignancies

| Drug/Drug | ABCB1 Efflux | |
|---|---|---|
| Combination | Blocker | Cancer Phenotype |
| Nelarabine | Mometasone furoate | T-lineage Acute lymphoblastic |
| | | Leukemia (T-ALL) |
| | | T-lineage lymphoblastic |
| | | Lymphoma (T-LL) |
| | | Peripheral T-cell lymphoma |
| | | Adult T-cell Leukemia |
| Daunorubicin | Mometasone furoate | Pre-B ALL |
| Doxorubicin | | Pre-B Lymphomas |
| Vincristine | | Large B-cell Lymphoma |
| | | Burkitts Lymphoma |
| | | B-cell ALL |
| Imatinib | Mometasone furoate | Philadelphia chromosome |
| | | positive ALL |
| | | Philadelphia chromosome |
| | | positive CML |

TABLE 3
Solid Tumor Malignancies

| Drug/Drug | ABCB1 Efflux | |
|---|---|---|
| Combination | Blocker | Cancer Phenotype |
| Daunorubicin | Mometasone furoate | Breast Cancer |
| Doxorubicin | | Ewing's Sarcoma |
| Vincristine | | Osteosarcoma |
| | | Undifferentiated high-grade |
| | | sarcomas |

All patents and publications referenced or mentioned herein are indicative of the levels of skill of those skilled in the art to which the invention pertains, and each of such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context of the use of the term clearly dictates otherwise. Thus, for example, a reference to “a compound” includes a plurality of compounds, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

### REFERENCES

1. Leonard G D, Fojo T, Bates S E: The role of ABC transporters in clinical practice. Oncologist 2003; 8:411-424.
2. Altenberg G A: Structure of multidrug-resistance proteins of the ATP-binding cassette (ABC) superfamily. Curr. Med. Che. Anti-Cancer Agents 2004; 4:53-62.
3. Wada M: Single nucleotide polymorphisms in ABCC2 and ABCB1 genes and their clinical impact in physiology and drug response. Cancer Lett 2006, 234:40-50.
4. Stycrynski J, Koltan A, Haus O, Wysocki M: Differential chemosensitivity in a child with congenital relapsing acute lymphoblastic leukemia. Pediatr Hemotol Oncol 2002: 19:355-360.
5. Kanerva J, Tiirikainen M. Makipernaa A. Riikonen P, Mottunen M, Salnd T T, et al: Multiple drug resistance mediated by P-lycoprotein is not a major factor in a slow response to therapy in childhood ALL. Pediatr Hentatol Oncol 1993; 15:11-21.
6. Bartlett N L, L.um 6L, Fisher G A, Brophy N A, Ehsan M N, Halsey J, et al: Phase I trial of doxorubicin with cyclosporine as a modulator of multidrug resistance. J. Clin Oncol 1994; 12:835-842.
7. Young S M, Bologa C, Prossnitz E R, Oprea Tl, Sklar L A. Edwards B S: High-throughput screening with HyperCyt flow cytometry to detect small molecule formylpeptidereceptor ligands. J. Biomol. Screen 2005; 10:374-382.
8. Legrand U, Perrot J Y, Simonin U, Baudard M, Mane J P: JC-1: a very sensitive fluorescence probe to test Pgp activity in adult acute myeloid leukemia Blood 2001; 97:502-508.
9. Kuhnel I M, Perrot J Y, Faussat A M, Marie J P, Schwaller M A: Functional assay of multidrug resistant cells using JC-1, a carbocyanine fluorescent probe. Leukemia 1997; 11:1147-1155.
10 Estes D A, Lovato D M, Khawaja H M, Winter S S, Larson R S: Genetic alterations determine chemotherapy resistance in childhood T-ALL: modelling in stage specific cell lines and correlation with diagnostic patient samples. Br J Haemarol 2007; 139:2030.
11. Winter S S, Jiang Z, Kha%vaja H M, Griffin T, Devidas M, Asselin B L, et al: Identification of genomic classifiers that distinguish induction failure in T-lineage acute lymphoblastic leukemia: a report from the Children's Oncology Group. Blood 2007; 110, 1429-1438,
12. Edwards B S, Bologa C, Young S M, Balakin K V, Prossnitz E R, Savchuck, N P, et al: Integration of virtual screening with high-throughput flow cytometry to identify novel small molecule formylpeptide receptor antagonists. Mol Pharmacol 2005; 68:1301-1310.
13. Winter S S, Holdsworth A T I; Devidas M, Raisch D W, Chauvenet A, Rav'sndranath Y, et al: Anti inetabolite-based therapy in childhood T-cell acute lymphoblastic leukemia: a report of POG study 9296. Pediatr Blood Cancer 2006; 46:179-186.
14. Jamroziak K, Robak T.: Pharmacogenomics of MDR1/ABCB1 gene: the influence on risk and clinical outcome of haematological malignancies. Hematology 2004; 9:91-105.
15. Emmet E A, Verweij C L, Durand D B, Higgins K M, Lacy E, Crabtree G R: Cyclosporin A specifically inhibits function of nuclear proteins involved in T cell activation. Science 1989; 246:1617-1620.
16. Vreugdenhil G, Raemaekers J M, van Dijke 133, de Pauw B E: Itraconazole and multidrug resistance: possible effects on remission rate and disease-free survival in acute leukemia. Ann Hematol 1993; 67:107-109.
17. Linn S C, van Kalken C K, van Tellingen O, van der Valk P, van Groeningen C J, Kuiper C M, et al Clinical and pharmacologic study of multidrug resistance reversal with vinblastine and bepridil J Clin Oncol 1994; 12:1312-819.
18. Petrini M, Caracciolo F, Carulli G, Conte A, Sabbatini A, Mattii L, et al: Vitamin D3 administration and, multidrug resistance in acute nonlymphoblastic leukemia. Acta Haematol 1993; 89:784-188.
19. Lacayo N J, Lum B L, Becton D L, Weinstein H, Ravindranath Y, Chang M N, et al: Pharmacokinetic interactions of cyclosporine with etoposide and mitoxaantrone in children with acute myeloid leukemia. Leukemia 2002; 76:920-927.
20. Poroikov V V, Filimonov D A: How to acquire new biological activities in old compounds by computer prediction. J. Comput Aided Mol Des. 2002; 16:819-824.
21. Takagi T, Ramachandran C, Bermejo M, Yamashita S, Yu L X, Amidon G L: A provisional biopharmaceutical classification of the top 200 oral drug products in the United States. Great Britain, Spain, and Japan. Mol Phann 2006; 3:631-643.
22. Wu C Y, Benet 1.Z: Predicting drug disposition via application of BCS: taans-pori/absorpticrnlelirnina [symbol] ion interplay and development of a biopharmaceutics chug disposition classification system. Pharm Res 2005; 22:1.1-23.
23. Nobili S. Landini 1, Giglioni B, Mini E: Pharmacological strategies for overcoming multidrug resistance. Curr Drug Targets 2006; 7:861-879.
24. Hegewisch-Becker S: M D R] reversal: criteria for clinical trials designed to overcome the multidrug resistance phenotype. Leukenia 1996; 10(supp] 3):S32-S38.
25. Gottesman M M, Fojo T, Bates S E: Multidrug resistance in cancer: role of KrF-dependent transporters. A'at Rev Cancer 2002; 2:48-58.
26. Robert J, Jarry C: Multidrug resistance reversal agents. J Med Chem 2003; 46:4805-4817.
27. Bates S F, Chen C, Robey R, King M, Figg W D, Fojo T. Reversal of multidrug resistance: lessons from clinical oncology. Novartis Found Symp 2002; 243:83-96; discussion 96-102, 180-105.

It will be apparent to those skilled in the art that various modifications and variations can be made in the devices and methods of the present disclosure. Other embodiments of the disclosure will be apparent to those skilled in the art from consideration of the specification and practice of the embodiments disclosed herein. It is intended that the specification and examples be considered as exemplary only.

1. A method of treating cancer in a patient or subject wherein drug resistance is mediated through ABCB1 transporter protein, said method comprising administering an effective amount of an inhibitor or efflux blocker of ABCB1 transporter protein to said patient or subject.

2. The method according to claim 1 wherein said inhibitor or efflux blocker is at least one compound selected from the group consisting of bepridil, nicardipine, propafenone, rescinnamine, mometasone furoate, 6-β-hydroxy mometosone furoate, ketoconazole, loxapine, pimozide, acacetin and cyclosporin A or a pharmaceutically acceptable salt thereof.

3. The method according to claim 1 or 2 wherein said cancer is a hematopoietic cancer or a tumor.

4. (canceled)

5. The method according to claim 1 wherein said cancer is a multiple drug resistant form (MDR) of cancer.

6. The method according to claim 1 wherein said cancer is Hodgkin's disease, non-Hodgkin's lymphoma, acute myelogenous leukemia, acute lymphocytic leukemia, acute promyelocytic leukemia (APL), acute T-cell lymphoblastic leukemia, R-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia, stem cell leukemia and metastases thereof.

7. The method according to claim 1 wherein said cancer is stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma and non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma.

8. The method according to claim 1 wherein said cancer is breast cancer, Ewing's sarcoma, osteosarcoma or an undifferentiated high-grade sarcoma, T-lineage acute lymphoblastic leukemia (T-ALL), T-lineage lymphoblastic lymphoma (T-LL), peripheral T-cell lymphoma, adult T-cell leukemia, pre-B ALL, pre-B lymphomas, large B-cell lymphoma, Burkitts lymphoma, B-cell ALL, Philadelphia chromosome positive ALL or Philadelphia chromosome positive CML.

9. The method according to claim 1 further comprising administering to said patient or subject an effective amount of at least one cancer drug in conjunction with said inhibitor.

10. (canceled)

11. (canceled)

12. The method according to claim 9 wherein said cancer drug is a compound or its pharmaceutically acceptable salt selected from the group consisting of Ara C, etoposide, doxorubicin, taxol, hydroxyurea, vincristine, cytoxan, mitomycin C, adriamycin, topotecan, campothecin, irinotecan, gemcitabine, cisplatin and mixtures thereof.

13. The method according to claim 9 wherein said cancer drug is a compound or its pharmaceutically acceptable salt selected from the group consisting of daunorubin, doxorubicin, epirubicin, idarubicin, valrubicin, vincristine, vinblastine, vindesine, vinorelbine, paclitaxel, docetoxel, etoposide, tenoposide, nelarabine, imatinib and mixtures thereof.

14. The method according to claim 9 wherein said cancer drug is a compound or its pharmaceutically acceptable salt selected from the group consisting of adriamycin, anastrozole, arsenic trioxide, asparaginase, azacytidine, BCG Live, bevacizumab, bexarotene capsules, bexarotene gel, bleomycin, bortezombi, busulfan intravenous, busulfan oral, calusterone, campothecin, capecitabine, carboplatin, carmustine, carmustine with polifeprosan 20 implant, celecoxib, cetuximab, chlorambucil, cisplatin, cladribine, clofarabine, cyclophosphamide, cytarabine, cytoxan, cytarabine liposomal, dacarbazine, dactinomycin, actinomycin D, dalteparin sodium, darbepoetin alfa, dasatinib, daunorubicin liposomal, daunorubicin, daunomycin, decitabine, denileukin, denileukin diftitox, dexrazoxane, dexrazoxane, docetaxel, doxorubicin, doxorubicin liposomal, dromostanolone propionate, eculizumab, Elliott's B Solution, epirubicin, epirubicin hcl, epoetin alfa, erlotinib, estramustine, etoposide phosphate, etoposide VP-16, exemestane, fentanyl citrate, filgrastim, floxuridine (intraarterial), fludarabine, fluorouracil 5-FU, fulvestrant, gefitinib, gemcitabine, gemcitabine hcl, gemicitabine, gemtuzumab ozogamicin, goserelin acetate, goserelin acetate, histrelin acetate, hydroxyurea, ibritumomab tiuxetan, idarubicin, ifosfamide, imatinib mesylate, interferon alfa 2a, interferon alfa-2b, irinotecan, lapatinib ditosylate, lenalidomide, letrozole, leucovorin, leuprolide acetate, levamisole, lomustine CCNU, meclorethamine, nitrogen mustard, megestrol acetate, melphalan L-PAM, mercaptopurine 6-MP, mesna, methotrexate, methoxsalen, mitomycin C, mitotane, mitoxantrone, nandrolone phenpropionate, nelarabine, nofetumomab, oprelvekin, oxaliplatin, paclitaxel, paclitaxel protein-bound particles, palifermin, pamidronate, panitumumab, pegademase, pegaspargase, pegfilgrastim, peginterferon alfa-2b, pemetrexed disodium, pentostatin, pipobroman, plicamycin, mithramycin, porfimer sodium, procarbazine, quinacrine, rasburicase, rituximab, sargramostim, sorafenib, streptozocin, sunitinib, sunitinib maleate, talc, tamoxifen, temozolomide, teniposide VM-26, testolactone, thalidomide, thioguanine 6-TG, thiotepa, topotecan, topotecan hcl, toremifene, tositumomab, tositumomab/I-131 tositumomab, trastuzumab, tretinoin ATRA, uracil mustard, valrubicin, vinblastine, vincristine, vinorelbine, vorinostat, zoledronate, zoledronic acid, or a pharmaceutically salt and mixtures thereof

15. The method according to 2 wherein said inhibitor is mometasone furoate.

16. (canceled)

17. (canceled)

18. (canceled)

19. The method according to claim 9 wherein said inhibitor and said cancer drug are administered simultaneously.

20. (canceled)

21. (canceled)

22. (canceled)

23. A method of inhibiting ABCB1 transporter protein in a patient or subject comprising administering to said patient or subject an effective amount of an ABCB1 inhibitor.

24. (canceled)

25. (canceled)

26. A pharmaceutical composition comprising an effective amount of an ABCB1 transporter protein inhibitor in combination with at least one additional anticancer agent, optionally in combination with a pharmaceutically acceptable carrier, additive or excipient.

27. The composition according to claim 26 wherein said ABCB1 inhibitor is selected from the group consisting of bepridil, nicardipine, propafenone, rescinnamine, mometasone furoate, 6-β-hydroxy mometosone furoate, ketoconazole, loxapine, pimozide, acacetin and cyclosporin A or a pharmaceutically acceptable salt thereof.

28. The composition according to claim 26 wherein said anticancer agent is a compound or its pharmaceutically acceptable salt selected from the group consisting of an antimetabolite, a topoisomerase I or a topoisomerase II inhibitor.

29. The composition according to claim 26 wherein said anticancer agent is selected from the group consisting of Ara C, etoposide, doxorubicin, taxol, hydroxyurea, vincristine, cytoxan, mitomycin C, adriamycin, topotecan, campothecin, irinotecan, gemcitabine and cisplatin or a pharmaceutically acceptable salt thereof.

30. The composition according to claim 26 wherein said anticancer agent is selected from the group consisting of daunorubin, doxorubicin, epirubicin, idarubicin, valrubicin, vincristine, vinblastine, vindesine, vinorelbine, paclitaxel, docetoxel, etoposide, tenoposide, nelarabine, imatinib or a pharmaceutically acceptable salt and mixtures thereof.

31. The composition according to claim 26 wherein said anticancer agent is a compound or its pharmaceutically acceptable salt selected from the group consisting of adriamycin, anastrozole, arsenic trioxide, asparaginase, azacytidine, BCG Live, bevacizumab, bexarotene capsules, bexarotene gel, bleomycin, bortezombi, busulfan intravenous, busulfan oral, calusterone, campothecin, capecitabine, carboplatin, carmustine, carmustine with polifeprosan 20 implant, celecoxib, cetuximab, chlorambucil, cisplatin, cladribine, clofarabine, cyclophosphamide, cytarabine, cytoxan, cytarabine liposomal, dacarbazine, dactinomycin, actinomycin D, dalteparin sodium, darbepoetin alfa, dasatinib, daunorubicin liposomal, daunorubicin, daunomycin, decitabine, denileukin, denileukin diftitox, dexrazoxane, dexrazoxane, docetaxel, doxorubicin, doxorubicin liposomal, dromostanolone propionate, eculizumab, Elliott's B Solution, epirubicin, epirubicin hcl, epoetin alfa, erlotinib, estramustine, etoposide phosphate, etoposide VP-16, exemestane, fentanyl citrate, filgrastim, floxuridine (intraarterial), fludarabine, fluorouracil 5-FU, fulvestrant, gefitinib, gemcitabine, gemcitabine hcl, gemicitabine, gemtuzumab ozogamicin, goserelin acetate, goserelin acetate, histrelin acetate, hydroxyurea, ibritumomab tiuxetan, idarubicin, ifosfamide, imatinib mesylate, interferon alfa 2a, interferon alfa-2b, irinotecan, lapatinib ditosylate, lenalidomide, letrozole, leucovorin, leuprolide acetate, levamisole, lomustine CCNU, meclorethamine, nitrogen mustard, megestrol acetate, melphalan L-PAM, mercaptopurine 6-MP, mesna, methotrexate, methoxsalen, mitomycin C, mitotane, mitoxantrone, nandrolone phenpropionate, nelarabine, nofetumomab, oprelvekin, oxaliplatin, paclitaxel, paclitaxel protein-bound particles, palifermin, pamidronate, panitumumab, pegademase, pegaspargase, pegfilgrastim, peginterferon alfa-2b, pemetrexed disodium, pentostatin, pipobroman, plicamycin, mithramycin, porfimer sodium, procarbazine, quinacrine, rasburicase, rituximab, sargramostim, sorafenib, streptozocin, sunitinib, sunitinib maleate, talc, tamoxifen, temozolomide, teniposide VM-26, testolactone, thalidomide, thioguanine 6-TG, thiotepa, topotecan, topotecan hcl, toremifene, tositumomab, tositumomab/I-131 tositumomab, trastuzumab, tretinoin ATRA, uracil mustard, valrubicin, vinblastine, vincristine, vinorelbine, vorinostat, zoledronate, zoledronic acid and mixtures thereof.

32. (canceled)

33. (canceled)

34. (canceled)

35. (canceled)

36. A method of treating cancer in a patient, reducing the likelihood that a cancer will recur or reducing the likelihood that a cancer will metastasize in a cancer patient comprising administering to said cancer patient an effective amount of a compound selected from the group consisting of inhibitor or efflux blocker is at least one compound selected from the group consisting of bepridil, nicardipine, propafenone, rescinnamine, mometasone furoate, 6-β-hydroxy mometosone furoate, ketoconazole, loxapine, pimozide, acacetin and cyclosporin A or a pharmaceutically acceptable salt thereof in combination with an anticancer drug to said patient.

37. The method according to claim 36 wherein said anticancer agent is a compound or its pharmaceutically acceptable salt selected from group consisting of Ara C, etoposide, doxorubicin, taxol, hydroxyurea, vincristine, cytoxan, mitomycin C, adriamycin, topotecan, campothecin, irinotecan, gemcitabine, cisplatin and mixtures thereof.

38. The method according to claim 36 wherein said cancer drug is a compound or its pharmaceutically acceptable salt selected from the group consisting of daunorubin, doxorubicin, epirubicin, idarubicin, valrubicin, vincristine, vinblastine, vindesine, vinorelbine, paclitaxel, docetoxel, etoposide, tenoposide, nelarabine, imatinib and mixtures thereof.

39. The method according to claim 36 wherein said cancer drug is a compound or its pharmaceutically acceptable salt selected from the group consisting of adriamycin, anastrozole, arsenic trioxide, asparaginase, azacytidine, BCG Live, bevacizumab, bexarotene capsules, bexarotene gel, bleomycin, bortezombi, busulfan intravenous, busulfan oral, calusterone, campothecin, capecitabine, carboplatin, carmustine, carmustine with polifeprosan 20 implant, celecoxib, cetuximab, chlorambucil, cisplatin, cladribine, clofarabine, cyclophosphamide, cytarabine, cytoxan, cytarabine liposomal, dacarbazine, dactinomycin, actinomycin D, dalteparin sodium, darbepoetin alfa, dasatinib, daunorubicin liposomal, daunorubicin, daunomycin, decitabine, denileukin, denileukin diftitox, dexrazoxane, dexrazoxane, docetaxel, doxorubicin, doxorubicin liposomal, dromostanolone propionate, eculizumab, Elliott's B Solution, epirubicin, epirubicin hcl, epoetin alfa, erlotinib, estramustine, etoposide phosphate, etoposide VP-16, exemestane, fentanyl citrate, filgrastim, floxuridine (intraarterial), fludarabine, fluorouracil 5-FU, fulvestrant, gefitinib, gemcitabine, gemcitabine hcl, gemicitabine, gemtuzumab ozogamicin, goserelin acetate, goserelin acetate, histrelin acetate, hydroxyurea, ibritumomab tiuxetan, idarubicin, ifosfamide, imatinib mesylate, interferon alfa 2a, interferon alfa-2b, irinotecan, lapatinib ditosylate, lenalidomide, letrozole, leucovorin, leuprolide acetate, levamisole, lomustine CCNU, meclorethamine, nitrogen mustard, megestrol acetate, melphalan L-PAM, mercaptopurine 6-MP, mesna, methotrexate, methoxsalen, mitomycin C, mitotane, mitoxantrone, nandrolone phenpropionate, nelarabine, nofetumomab, oprelvekin, oxaliplatin, paclitaxel, paclitaxel protein-bound particles, palifermin, pamidronate, panitumumab, pegademase, pegaspargase, pegfilgrastim, peginterferon alfa-2b, pemetrexed disodium, pentostatin, pipobroman, plicamycin, mithramycin, porfimer sodium, procarbazine, quinacrine, rasburicase, rituximab, sargramostim, sorafenib, streptozocin, sunitinib, sunitinib maleate, talc, tamoxifen, temozolomide, teniposide VM-26, testolactone, thalidomide, thioguanine 6-TG, thiotepa, topotecan, topotecan hcl, toremifene, tositumomab, tositumomab/I-131 tositumomab, trastuzumab, tretinoin ATRA, uracil mustard, valrubicin, vinblastine, vincristine, vinorelbine, vorinostat, zoledronate, zoledronic acid, and mixtures thereof.

40. The method according to claim 36 wherein said cancer is cancer is Hodgkin's disease, non-Hodgkin's lymphoma, acute myelogenous leukemia, acute lymphocytic leukemia, acute promyelocytic leukemia (APL), acute T-cell lymphoblastic leukemia, R-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia, stem cell leukemia and metastases thereof.

41. The method according claim 36 wherein said cancer is stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma and non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma.

42. The method according to claim 36 wherein said cancer is breast cancer, Ewing's sarcoma, osteosarcoma or an undifferentiated high-grade sarcoma, T-lineage acute lymphoblastic leukemia (T-ALL), T-lineage lymphoblastic lymphoma (T-LL), peripheral T-cell lymphoma, adult T-cell leukemia, pre-B ALL, pre-B lymphomas, large B-cell lymphoma, Burkitts lymphoma, B-cell ALL, Philadelphia chromosome positive ALL or Philadelphia chromosome positive CML.

43. A method to measure the ability of test compounds to inhibit the function of ABCB1 and ABCG2 transporters, comprising the steps of:
labeling Jurkat-DNR cells with an effective amount of FarRed DDAO CellTrace SE (Invitrogen);
washing said Jurkat-DNR cells;
combining said Jurkat-DNR cells with unlabeled IgMXP3 cells in an assay buffer to form a cell suspension, allowing the label to bind covalently to amine groups;
adding an effective amount of solution comprising JC1 substrate solution to the cell suspension;
subsequently dispensing cells from the cell suspension into well plates;
adding test and control compounds to the cell suspension in the wells;
incubating the test and control compounds in the cell suspension in the wells; and
delivering the cell suspension from the wells to a flow cytometer to determine transporter protein pump activity, and
determining whether or not a test compound is an inhibitor of ABCB1 and/or ABCG2.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)
